(19) United States
(12) Patent Application Publication
(10) Pub. No.: US 2023/0089534 A1
(43) Pub. Date: Mar. 23, 2023

(54) PHENOLIC COMPOSITIONS FOR MALODOR REDUCTION

(71) Applicant: Milliken & Company, Spartanburg, SC (US)

(72) Inventors: Wesley Freund, Simpsonville, SC (US); Nicholas L'Amoreaux, Greenville, SC (US)

(21) Appl. No.: 17/939,556

(22) Filed: Sep. 7, 2022

Related U.S. Application Data
(60) Provisional application No. 63/242,214, filed on Sep. 9, 2021.

(51) Int. Cl. C11D 3/20; C07C 69/732; C11D 11/00; C11D 3/00

(52) U.S. Cl. CPC C11D 3/2093; C07C 69/732; C11D 11/0017; C11D 3/0068; C11D 3/0084

(57) ABSTRACT

Disclosed herein are phenolic compositions and laundry care compositions comprising such phenolic compositions. The laundry care compositions may reduce malodor of fabrics or articles laundered with the same.

### CROSS-REFERENCE TO RELATED PATENT APPLICATIONS

This application claims, pursuant to 35 U.S.C. § 119(e), priority to and the benefit of the filing date of U.S. Patent Application No. 63/242,214, which was filed on Sep. 9, 2021, the contents of which are hereby incorporated by reference.

### FIELD OF THE INVENTION

The present disclosure relates to phenolic compositions for use as antioxidants in fabric treatment compositions for reducing malodor.

### BACKGROUND

Malodor is a lingering issue in laundry. There are a number of sources of malodor causing materials on textiles. One source of malodor comes from the fact that as consumers move toward the use of less water, shorter wash cycles and lower wash water temperatures, soils are not as efficiently removed from the clothes during the wash cycle and remain on the surface of the fabrics. These soils that remain on the fabric service may consist of human sebum and other oils that are prone to autoxidation and breakdown into volatile, malodorous compounds.

Manufacturers are continuously seeking ways to reduce this malodor. One approach involves the use of antioxidants in the laundry that are deposited onto clothing during the laundering. These antioxidants reduce the rate of autoxidation of the oils, and in turn prevent the generation of malodorous compounds.

There remains a need for improved antioxidant compositions for reducing malodor in fabrics and textiles. There remains a need for improved methods of preventing oxidation of sebum and other oils in fabric and textiles. There remains a need for improved antioxidant compositions that effectively reduce and block malodor over increased periods of time. There remains a need for improved antioxidant compositions that effectively reduce and block malodor when applied at lower concentrations. There remains a need for improved antioxidant compositions that are more readily formulated into detergent compositions. There remains a need for improved antioxidant compositions that are more readily transferred to fabrics and textiles when laundered.

### SUMMARY

In accordance with the purposes of the disclosed materials and methods, as embodied and broadly described herein, the disclosed subject matter, in one aspect, relates to compounds, compositions and methods of making and using compounds and compositions.

Additional advantages will be set forth in part in the description that follows, and in part will be obvious from the description, or may be learned by practice of the aspects described below. The advantages described below will be realized and attained by means of the elements and combinations particularly pointed out in the appended claims. It is to be understood that both the foregoing general description and the following detailed description are exemplary and explanatory only and are not restrictive.

The details of one or more embodiments are set forth in the descriptions below. Other features, objects, and advantages will be apparent from the description and from the claims.

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes-, from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers, or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

As used herein the phrase “fabric treatment composition” includes compositions and formulations designed for treating fabrics, including garments, or other textiles. Such compositions include, but are not limited to, laundry cleaning compositions and detergents, fabric softening compositions, fabric enhancing compositions, fabric freshening compositions, laundry prewash, laundry pretreat, laundry additives, spray products, dry cleaning agent or composition, laundry wash additives, post-rinse fabric treatments, ironing aid, unit dose formulation, delayed delivery formulation, detergent contained on or in a porous substrate or nonwoven sheet, and other suitable forms that may be apparent to one skilled in the art in view of the teachings herein. Such compositions may be used as a pre-laundering treatment, a post-laundering treatment, or may be added during the wash cycle of the laundering operation.

As used herein, “liquid” includes free-flowing liquids, as well as pastes, gels, foams, and mousses. Non-limiting examples of liquids include light-duty and heavy-duty liquid detergent compositions, fabric enhancers, detergent gels commonly used for laundry, bleach, and laundry additives. Gases, e.g., suspended bubbles, or solids, e.g., particles, may be included within the liquids.

As used herein, a “granule” and a “particle” refer to a volume of solid, or sufficiently solid, material that has finite mass. Granules and particles may be free-flowing or suspended within a secondary composition. Free-flowing particles may be similar to those commercially available under the tradename UNSTOPABLES® from The Procter & Gamble Company, Cincinnati, Ohio, United States.

The terms “substantially free of” or “substantially free from” may be used herein. This means that the indicated material is at the very minimum not deliberately added to the composition to form part of it, or, preferably, is not present at analytically detectable levels. It is meant to include compositions whereby the indicated material is present only as an impurity in one of the other materials deliberately included. The indicated material may be present, if at all, at a level of less than 10%, or less than 1%, or less than 0.1%, or less than 0.01%, or even 0%, by weight of the composition.

As used herein, the phrases “sufficiently solid” and “solid” mean the material is capable of maintaining its shape without significant deformation when free-standing at room temperature. A “solid” as used herein may include, but is not limited to, granules, particles, powders, agglomerates, micro-capsules, flakes, noodles, pearlized balls, and mixtures thereof.

As used herein, the phrase “water-soluble”, “water-soluble material,” “water-soluble carrier material,” means that the material or carrier material is soluble or dispersible in water, and preferably has a water-solubility of at least 50%, preferably at least 75% or even at least 95%, as measured by the method set out hereafter using a glass-filter with a maximum pore size of 20 microns: 50 grams±0.1 gram of the material and/or carrier material is added in a pre-weighed 400 mL beaker and 245 mL±1 mL of distilled water is added. This is stirred vigorously on a magnetic stirrer set at 600 rpm, for 30 minutes. Then, the mixture is filtered through a sintered-glass filter with a pore size as defined above (max. 20 micron). The steps are performed at ambient conditions. “Ambient conditions” as used herein means 23° C.±1.0° C. and a relative humidity of 50%±2%. The water is dried off from the collected filtrate by any conventional method, and the weight of the remaining material is determined (which is the dissolved or dispersed fraction). Then, the percentage solubility or dispersibility can be calculated.

Unless otherwise noted, all component/ingredient or composition levels are in reference to the active portion of that component/ingredient or composition, and are exclusive of impurities, for example, residual solvents or by-products, which may be present in commercially available sources of such components/ingredients or compositions.

Disclosed herein are phenolic compositions for use as antioxidants in fabric treatment compositions. The phenolic compositions include at least a compound of Formula (I) and a compound of Formula (II):

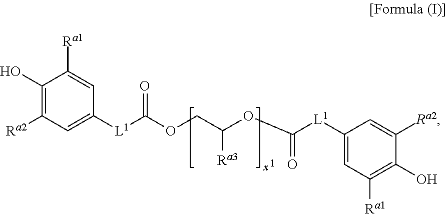

wherein L¹ is selected from null, —CH₂—, —CH₂CH₂—, and —CH₂CH₂CH₂—,
x¹ is an integer from 2-50;
R^{a1} is a C₁₋₈alkyl group;
R^{a2} is a C₁₋₈alkyl group;
each R^{a3} is independently selected from the group consisting of H, CH₃, and CH₂CH₃;

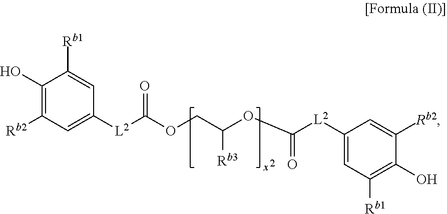

wherein L² is selected from null, —CH₂—, —CH₂CH₂—, and —CH₂CH₂CH₂—,
x² is an integer from 2-50; provided that x¹ and x² are not the same;
R^{b1} is a C₁₋₈alkyl group;
R^{b2} is a C₁₋₈alkyl group; and
each R^{b3} is independently selected from the group consisting of H, CH₃, and CH₂CH₃.

In the context of Formula (I) and Formula (II) and the formula for the other phenolic compounds that follow, the term “null” is meant to convey that the phenol moiety is directly bonded to the carbonyl carbon atom via the carbon atom of the phenol moiety that is located in the para-position relative to the hydroxy group of the phenol moiety. The use of brackets for the —[CH₂—CHR³—O]— group does not imply that each R³ group is necessarily the same. Unless specified to the contrary, a given compound can have the same or different R³ substituents at each repeating unit of the chain. For example, in some embodiments of the compound of Formula (I) x=4. In some such embodiments, R^{a3} can in each case be hydrogen, while in other cases two R^{a3} groups can be hydrogen, and the other two R^{a3} groups can be methyl.

Without wishing to be bound by theory, it is believed the use of multiple phenolic antioxidants impart favorable antioxidant strength, as well as stability, relative to the use of a single phenolic compound as an antioxidant.

In some embodiments, the phenolic compositions can further include a compound of Formula (III):

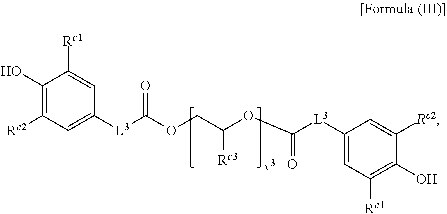

wherein L³ is selected from null, —CH₂—, —CH₂CH₂—, and —CH₂CH₂CH₂—,
x³ is an integer from 2-50; provided that none of x¹, x², and x³ are the same;
R^{c1} is a C₁₋₈alkyl group;
R^{c2} is a C₁₋₈alkyl group; and
each R^{c3} is independently selected from the group consisting of H, CH₃, and CH₂CH₃.

In some embodiments, the phenolic compositions can further include a compound of Formula (IV):

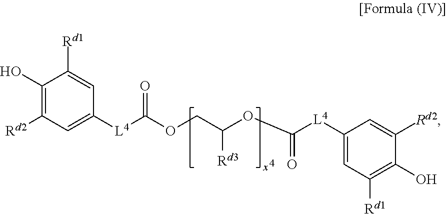

wherein L⁴ is selected from null, —CH₂—, —CH₂CH₂—, and —CH₂CH₂CH₂—,
x⁴ is an integer from 2-50; provided that none of x¹, x², x³, and x⁴ are the same;
R^{d1} is a C₁₋₈alkyl group;
R^{d2} is a C₁₋₈alkyl group; and
each R^{d3} is independently selected from the group consisting of H, CH₃, and CH₂CH₃.

In some embodiments, the phenolic compositions can further include a compound of Formula (V):

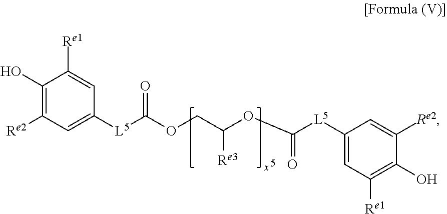

wherein L⁵ is selected from null, —CH₂—, —CH₂CH₂—, and —CH₂CH₂CH₂—,
x⁵ is an integer from 2-50; provided that none of x¹, x², x³, x⁴, and x⁵ are the same;
R^{e1} is a C₁₋₈alkyl group;
R^{e2} is a C₁₋₈alkyl group; and
each R^{e3} is independently selected from the group consisting of H, CH₃, and CH₂CH₃.

In some embodiments, the phenolic compositions can further include a compound of Formula (VI):

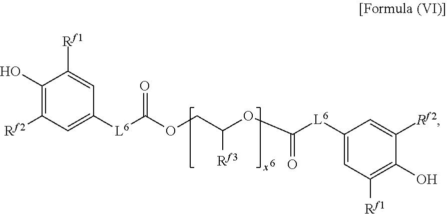

wherein L⁶ is selected from null, —CH₂—, —CH₂CH₂—, and —CH₂CH₂CH₂—,
x⁶ is an integer from 2-50; provided that none of x¹, x², x³, x⁴, x⁵, and x⁶ are the same;
R^{f1} is a C₁₋₈alkyl group;
R^{f2} is a C₁₋₈alkyl group; and
each R³ is independently selected from the group consisting of H, CH₃, and CH₂CH₃.

In some embodiments, the phenolic compositions can further include a compound of Formula (VII):

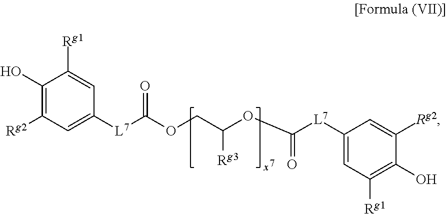

wherein L⁷ is selected from null, —CH₂—, —CH₂CH₂—, and —CH₂CH₂CH₂—,
x⁷ is an integer from 2-50; provided that none of x¹, x², x³, x⁴, x⁵, x⁶, and x⁷ are the same;
R^{g1} is a C₁₋₈alkyl group;
R^{g2} is a C₁₋₈alkyl group; and
each R^{g3} is independently selected from the group consisting of H, CH₃, and CH₂CH₃.

In some embodiments, the phenolic compositions can further include a compound of Formula (VIII):

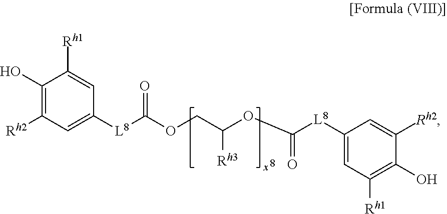

wherein L⁸ is selected from null, —CH₂—, —CH₂CH₂—, and —CH₂CH₂CH₂—,
x⁸ is an integer from 2-50; provided that none of x¹, x², x³, x⁴, x⁵, x⁶, x⁷, and x⁸ are the same;
R^{h1} is a C₁₋₈alkyl group;
R^{h2} is a C₁₋₈alkyl group; and
each R^{h3} is independently selected from the group consisting of H, CH₃, and CH₂CH₃.

In some embodiments, it is preferred that each of the L substituents (i.e., L¹, L², L³, L⁴, L⁵, L⁶, L⁷, and L⁸) is the same. For example, each L substituent can be null, or each L substituent can be —CH₂—, or each L substituent can be —CH₂CH₂—, or each L substituent can be —CH₂CH₂CH₂—. In preferred embodiments, each L substituent is —CH₂CH₂—.

In certain embodiments, it is preferred that the R¹ substituents (i.e., R^{a1}, R^{b1}, R^{c1}, R^{d1}, R^{e1}, R^{f1}, R^{g1}, and R^{h1}) are not the same as the R² substituents (i.e., R^{a2}, R^{b2}, R^{c2}, R^{d2}, R^{e2}, R^{f2}, R^{g2}, and R^{h2}). For example, each of the R¹ substituents can be a tert-butyl group, while each of the R² substituents can be a linear C₁₋₈alkyl group (i.e., methyl, ethyl, n-propyl, n-butyl, n-pentyl, n-hexyl, n-heptyl, or n-octyl). In certain embodiments, it is preferred than each of the R¹ substituents is a tert-butyl group, and each of the R² substituents is a methyl group, or each of the R² substituents is an ethyl group.

In some embodiments, it is preferred that each of the R³ substituents (i.e., R^{a3}, R^{b3}, R^{c3}, R^{d3}, R^{e3}, R^{f3}, R^{g3}, and R^{h3}) are the same. For example, each of the R³ substituents can be H, or each of the R³ substituents can be CH₃. In other embodiments, at least one of the R³ substituents is H and at least one of the R³ substituents is CH₃.

For embodiments in which each of the R¹ substituents are the same, each of the R² substituents are the same, each of the R³ substituents are the same, and each of the L substituents are the same, the phenolic compositions can be described as a plurality of compounds having the general formula:

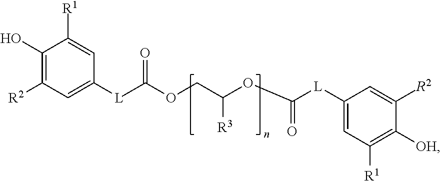

wherein Lis selected from null, —CH₂—, —CH₂CH₂—, and —CH₂CH₂CH₂—, R¹ is a C₁₋₈alkyl group;
R² is a C₁₋₈alkyl group;
R³ is H, CH₃, or CH₂CH₃; and
n is greater than 2 and less than 50, preferably greater than 3 and less than 50, greater than 4 and less than 25, greater than 4 and less than 10, greater than 5 and less than 15, greater than 8 and less than 20, or greater than 12 and less than 25.

The ‘n’ value of a given phenolic composition may be determined by HPLC analysis with a UV/Vis detector. This type of analysis is the preferred method to quantify the levels of each component of the polymeric distribution, as it will give the most accurate, reproducible results with phenolic compositions of this type. In other embodiments, mass spectrometry data may also be used.

In some embodiments, the compound of Formula I is characterized by x¹=4, the compound of Formula II is characterized by x²=3. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=4, the compound of Formula II is characterized by x²=3, and the compound of Formula III is characterized by x³=5. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=5, the compound of Formula II is characterized by x²=4. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=5, the compound of Formula II is characterized by x²=4, and the compound of Formula III is characterized by x³=6. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=6, the compound of Formula II is characterized by x²=5. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=6, the compound of Formula II is characterized by x²=5, and the compound of Formula III is characterized by x³=7. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=7, the compound of Formula II is characterized by x²=6. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=7, the compound of Formula II is characterized by x²=6, and the compound of Formula III is characterized by x³=8. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=8, the compound of Formula II is characterized by x²=7. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=8, the compound of Formula II is characterized by x²=7, and the compound of Formula III is characterized by x³=9. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=9, the compound of Formula II is characterized by x²=7. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=9, the compound of Formula II is characterized by x²=7, and the compound of Formula III is characterized by x³=10. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=10, the compound of Formula II is characterized by x²=9 The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=10, the compound of Formula II is characterized by x²=9, and the compound of Formula III is characterized by x³=11. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=11, the compound of Formula II is characterized by x²=10. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=11, the compound of Formula II is characterized by x²=10, and the compound of Formula III is characterized by x³=12. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=12, the compound of Formula II is characterized by x²=11. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=12, the compound of Formula II is characterized by x²=11, and the compound of Formula III is characterized by x³=13. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=13, the compound of Formula II is characterized by x²=12. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=13, the compound of Formula II is characterized by x²=12, and the compound of Formula III is characterized by x³=14. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=14, the compound of Formula II is characterized by x²=13. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=14, the compound of Formula II is characterized by x²=13, and the compound of Formula III is characterized by x³=15. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=15, the compound of Formula II is characterized by x²=14. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=15, the compound of Formula II is characterized by x²=14, and the compound of Formula III is characterized by x³=16. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=16, the compound of Formula II is characterized by x²=15 The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=16 the compound of Formula II is characterized by x²=15, and the compound of Formula III is characterized by x³=17. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In some embodiments, the compound of Formula I is characterized by x¹=17, the compound of Formula II is characterized by x²=16. The compound of Formula I may be present in the phenolic composition in an amount from 15-75%, relative to the total mass of the phenolic composition, and the compound of Formula II may be present in an amount from 15-75%, relative to the total mass of the phenolic composition.

In certain embodiments, the compound of Formula I is characterized by x¹=17, the compound of Formula II is characterized by x²=16, and the compound of Formula III is characterized by x³=18. The compound of Formula I may be present in the phenolic composition in an amount from 15-75%, relative to the total mass of the phenolic composition, the compound of Formula II may be present in an amount from 15-75%, relative to the total mass of the phenolic composition, and the compound of Formula III may be present in an amount from 15-75%, relative to the total mass of the phenolic composition.

In certain embodiments, the compound of Formula I is characterized by x¹=3, the compound of Formula II is characterized by x²=4, the compound of Formula III is characterized by x³=5, the compound of Formula IV is characterized by x⁴=6, the compound of Formula V is characterized by x⁵=7, the compound of Formula VI is characterized by x⁶=8, the compound of Formula VII is characterized by x⁷=9, and the compound of Formula VIII is characterized by x⁸=10. These compounds may be present in the phenolic compositions as shown in the table below (expressed as weight percent relative to the total mass of the phenolic composition):

| I | II | III | IV | V | VI | VII | VIII |
|---|---|---|---|---|---|---|---|
| 25-75% | 25-75% | 0-50% | 0-50% | 0-50% | 0-50% | 0-50% | 0-50% |
| 25-75% | 25-75% | 25-75% | 0-25% | 0-25% | 0-25% | 0-25% | 0-25% |
| 0-50% | 25-75% | 25-75% | 0-50% | 0-50% | 0-50% | 0-50% | 0-50% |
| 0-25% | 25-75% | 25-75% | 25-75% | 0-25% | 0-25% | 0-25% | 0-25% |
| 0-50% | 0-50% | 25-75% | 25-75% | 0-50% | 0-50% | 0-50% | 0-50% |
| 0-25% | 0-25% | 25-75% | 25-75% | 25-75% | 0-25% | 0-25% | 0-25% |
| 0-50% | 0-50% | 0-50% | 25-75% | 25-75% | 0-50% | 0-50% | 0-50% |
| 0-25% | 0-25% | 0-25% | 25-75% | 25-75% | 25-75% | 0-25% | 0-25% |
| 0-50% | 0-50% | 0-50% | 0-50% | 25-75% | 25-75% | 0-50% | 0-50% |
| 0-25% | 0-25% | 0-25% | 0-25% | 25-75% | 25-75% | 25-75% | 0-25% |
| 0-50% | 0-50% | 0-50% | 0-50% | 0-50% | 25-75% | 25-75% | 0-50% |
| 0-25% | 0-25% | 0-25% | 0-25% | 0-25% | 25-75% | 25-75% | 25-75% |
| 15-40% | 15-40% | 15-35% | 5-15% | <10% | <5% | <3% | <3% |
| <5% | 5-15% | 10-25% | 15-35% | 15-35% | 5-25% | 2-10% | <5% |
| <3% | <1% | <5% | 5-15% | 8-20% | 10-25% | 10-25% | 9-20% |
| 7-20% | 7-20% | 10-25% | 10-20% | 5-15% | 2-15% | 1-5% | <5% |
| 5-15% | 5-15% | 5-10% | <5% | <5% | 5-10% | 5-15% | 5-15% |
| 15-50% | 15-50% | 15-50% | 15-50% | 0-55% | 0-55% | 0-55% | 0-55% |
| 0-55% | 15-50% | 15-50% | 15-50% | 15-50% | 0-55% | 0-55% | 0-55% |
| 0-55% | 0-55% | 15-50% | 15-50% | 15-50% | 15-50% | 0-55% | 0-55% |
| 0-55% | 0-55% | 0-55% | 15-50% | 15-50% | 15-50% | 15-50% | 0-55% |
| 0-55% | 0-55% | 0-55% | 0-55% | 15-50% | 15-50% | 15-50% | 15-50% |
| 15-40% | 15-40% | 15-40% | 15-40% | 15-40% | 0-25% | 0-25% | 0-25% |
| 0-25% | 15-40% | 15-40% | 15-40% | 15-40% | 15-40% | 0-25% | 0-25% |
| 0-25% | 0-25% | 15-40% | 15-40% | 15-40% | 15-40% | 15-40% | 0-25% |
| 0-25% | 0-25% | 0-25% | 15-40% | 15-40% | 15-40% | 15-40% | 15-40% |

In certain embodiments, the compound of Formula I is characterized by x¹=6, the compound of Formula II is characterized by x²=7, the compound of Formula III is characterized by x³=8, the compound of Formula IV is characterized by x⁴=9, the compound of Formula V is characterized by x⁵=10, the compound of Formula VI is characterized by x⁶=11, the compound of Formula VII is characterized by x⁷=12, and the compound of Formula VIII is characterized by x⁸=13. These compounds may be present in the phenolic compositions as shown in the table below (expressed as weight percent relative to the total mass of the phenolic composition):

| I | II | III | IV | V | VI | VII | VIII |
|---|---|---|---|---|---|---|---|
| 25-75% | 25-75% | 0-50% | 0-50% | 0-50% | 0-50% | 0-50% | 0-50% |
| 25-75% | 25-75% | 25-75% | 0-25% | 0-25% | 0-25% | 0-25% | 0-25% |
| 0-50% | 25-75% | 25-75% | 0-50% | 0-50% | 0-50% | 0-50% | 0-50% |
| 0-25% | 25-75% | 25-75% | 25-75% | 0-25% | 0-25% | 0-25% | 0-25% |
| 0-50% | 0-50% | 25-75% | 25-75% | 0-50% | 0-50% | 0-50% | 0-50% |
| 0-25% | 0-25% | 25-75% | 25-75% | 25-75% | 0-25% | 0-25% | 0-25% |
| 0-50% | 0-50% | 0-50% | 25-75% | 25-75% | 0-50% | 0-50% | 0-50% |
| 0-25% | 0-25% | 0-25% | 25-75% | 25-75% | 25-75% | 0-25% | 0-25% |
| 0-50% | 0-50% | 0-50% | 0-50% | 25-75% | 25-75% | 0-50% | 0-50% |
| 0-25% | 0-25% | 0-25% | 0-25% | 25-75% | 25-75% | 25-75% | 0-25% |
| 0-50% | 0-50% | 0-50% | 0-50% | 0-50% | 25-75% | 25-75% | 0-50% |
| 0-25% | 0-25% | 0-25% | 0-25% | 0-25% | 25-75% | 25-75% | 25-75% |
| 15-40% | 15-40% | 15-35% | 5-15% | <10% | <5% | <3% | <3% |
| <5% | 5-15% | 10-25% | 15-35% | 15-35% | 5-25% | 2-10% | <5% |
| <3% | <1% | <5% | 5-15% | 8-20% | 10-25% | 10-25% | 9-20% |
| 7-20% | 7-20% | 10-25% | 10-20% | 5-15% | 2-15% | 1-5% | <5% |
| 5-15% | 5-15% | 5-10% | <5% | <5% | 5-10% | 5-15% | 5-15% |
| 15-50% | 15-50% | 15-50% | 15-50% | 0-55% | 0-55% | 0-55% | 0-55% |
| 0-55% | 15-50% | 15-50% | 15-50% | 15-50% | 0-55% | 0-55% | 0-55% |
| 0-55% | 0-55% | 15-50% | 15-50% | 15-50% | 15-50% | 0-55% | 0-55% |
| 0-55% | 0-55% | 0-55% | 15-50% | 15-50% | 15-50% | 15-50% | 0-55% |
| 0-55% | 0-55% | 0-55% | 0-55% | 15-50% | 15-50% | 15-50% | 15-50% |
| 15-40% | 15-40% | 15-40% | 15-40% | 15-40% | 0-25% | 0-25% | 0-25% |
| 0-25% | 15-40% | 15-40% | 15-40% | 15-40% | 15-40% | 0-25% | 0-25% |
| 0-25% | 0-25% | 15-40% | 15-40% | 15-40% | 15-40% | 15-40% | 0-25% |
| 0-25% | 0-25% | 0-25% | 15-40% | 15-40% | 15-40% | 15-40% | 15-40% |

In certain embodiments, the compound of Formula I is characterized by x¹=10, the compound of Formula II is characterized by x²=11, the compound of Formula III is characterized by x³=12, the compound of Formula IV is characterized by x⁴=13, the compound of Formula V is characterized by x⁵=14, the compound of Formula VI is characterized by x⁶=15, the compound of Formula VII is characterized by x⁷=16, and the compound of Formula VIII is characterized by x⁸=17. These compounds may be present in the phenolic compositions as shown in the table below (expressed as weight percent relative to the total mass of the phenolic composition):

| I | II | III | IV | V | VI | VII | VIII |
|---|---|---|---|---|---|---|---|
| 25-75% | 25-75% | 0-50% | 0-50% | 0-50% | 0-50% | 0-50% | 0-50% |
| 25-75% | 25-75% | 25-75% | 0-25% | 0-25% | 0-25% | 0-25% | 0-25% |
| 0-50% | 25-75% | 25-75% | 0-50% | 0-50% | 0-50% | 0-50% | 0-50% |
| 0-25% | 25-75% | 25-75% | 25-75% | 0-25% | 0-25% | 0-25% | 0-25% |
| 0-50% | 0-50% | 25-75% | 25-75% | 0-50% | 0-50% | 0-50% | 0-50% |
| 0-25% | 0-25% | 25-75% | 25-75% | 25-75% | 0-25% | 0-25% | 0-25% |
| 0-50% | 0-50% | 0-50% | 25-75% | 25-75% | 0-50% | 0-50% | 0-50% |
| 0-25% | 0-25% | 0-25% | 25-75% | 25-75% | 25-75% | 0-25% | 0-25% |
| 0-50% | 0-50% | 0-50% | 0-50% | 25-75% | 25-75% | 0-50% | 0-50% |
| 0-25% | 0-25% | 0-25% | 0-25% | 25-75% | 25-75% | 25-75% | 0-25% |
| 0-50% | 0-50% | 0-50% | 0-50% | 0-50% | 25-75% | 25-75% | 0-50% |
| 0-25% | 0-25% | 0-25% | 0-25% | 0-25% | 25-75% | 25-75% | 25-75% |
| 15-40% | 15-40% | 15-35% | 5-15% | <10% | <5% | <3% | <3% |
| <5% | 5-15% | 10-25% | 15-35% | 15-35% | 5-25% | 2-10% | <5% |
| <3% | <1% | <5% | 5-15% | 8-20% | 10-25% | 10-25% | 9-20% |
| 7-20% | 7-20% | 10-25% | 10-20% | 5-15% | 2-15% | 1-5% | <5% |
| 5-15% | 5-15% | 5-10% | <5% | <5% | 5-10% | 5-15% | 5-15% |
| 15-50% | 15-50% | 15-50% | 15-50% | 0-55% | 0-55% | 0-55% | 0-55% |
| 0-55% | 15-50% | 15-50% | 15-50% | 15-50% | 0-55% | 0-55% | 0-55% |
| 0-55% | 0-55% | 15-50% | 15-50% | 15-50% | 15-50% | 0-55% | 0-55% |
| 0-55% | 0-55% | 0-55% | 15-50% | 15-50% | 15-50% | 15-50% | 0-55% |
| 0-55% | 0-55% | 0-55% | 0-55% | 15-50% | 15-50% | 15-50% | 15-50% |
| 15-40% | 15-40% | 15-40% | 15-40% | 15-40% | 0-25% | 0-25% | 0-25% |
| 0-25% | 15-40% | 15-40% | 15-40% | 15-40% | 15-40% | 0-25% | 0-25% |
| 0-25% | 0-25% | 15-40% | 15-40% | 15-40% | 15-40% | 15-40% | 0-25% |
| 0-25% | 0-25% | 0-25% | 15-40% | 15-40% | 15-40% | 15-40% | 15-40% |

Exemplary phenolic compounds that may be used in the phenolic compositions include:

| Cpd. | R¹ | R² | L | R³ | X |
|---|---|---|---|---|---|
| 2-A | C(CH3)₃ | CH₃ | -CH₂CH₂- | H | 2 |
| 2-B | C(CH3)₃ | CH₂CH₃ | -CH₂CH₂- | H | 2 |
| 2-C | C(CH3)₃ | CH₃ | -CH₂CH₂- | CH₃ | 2 |
| 2-D | C(CH3)₃ | CH₂CH₃ | -CH₂CH₂- | CH₃ | 2 |
| 3-A | C(CH3)₃ | CH₃ | -CH₂CH₂- | H | 3 |
| 3-B | C(CH3)₃ | CH₂CH₃ | -CH₂CH₂- | H | 3 |
| 3-C | C(CH3)₃ | CH₃ | -CH₂CH₂- | CH₃ | 3 |
| 3-D | C(CH3)₃ | CH₂CH₃ | -CH₂CH₂- | CH₃ | 3 |
| 4-A | C(CH3)₃ | CH₃ | -CH₂CH₂- | H | 4 |
| 4-B | C(CH3)₃ | CH₂CH₃ | -CH₂CH₂- | H | 4 |
| 4-C | C(CH3)₃ | CH₃ | -CH₂CH₂- | CH₃ | 4 |
| 4-D | C(CH3)₃ | CH₂CH₃ | -CH₂CH₂- | CH₃ | 4 |
| 5-A | C(CH3)₃ | CH₃ | -CH₂CH₂- | H | 5 |
| 5-B | C(CH3)₃ | CH₂CH₃ | -CH₂CH₂- | H | 5 |
| 5-C | C(CH3)₃ | CH₃ | -CH₂CH₂- | CH₃ | 5 |
| 5-D | C(CH3)₃ | CH₂CH₃ | -CH₂CH₂- | CH₃ | 5 |
| 6-A | C(CH3)₃ | CH₃ | -CH₂CH₂- | H | 6 |
| 6-B | C(CH3)₃ | CH₂CH₃ | -CH₂CH₂- | H | 6 |
| 6-C | C(CH3)₃ | CH₃ | -CH₂CH₂- | CH₃ | 6 |
| 6-D | C(CH3)₃ | CH₂CH₃ | -CH₂CH₂- | CH₃ | 6 |
| 7-A | C(CH3)₃ | CH₃ | -CH₂CH₂- | H | 7 |
| 7-B | C(CH3)₃ | CH₂CH₃ | -CH₂CH₂- | H | 7 |
| 7-C | C(CH3)₃ | CH₃ | -CH₂CH₂- | CH₃ | 7 |
| 7-D | C(CH3)₃ | CH₂CH₃ | -CH₂CH₂- | CH₃ | 7 |
| 8-A | C(CH3)₃ | CH₃ | -CH₂CH₂- | H | 8 |
| 8-B | C(CH3)₃ | CH₂CH₃ | -CH₂CH₂- | H | 8 |
| 8-C | C(CH3)₃ | CH₃ | -CH₂CH₂- | CH₃ | 8 |
| 8-D | C(CH3)₃ | CH₂CH₃ | -CH₂CH₂- | CH₃ | 8 |
| 9-A | C(CH3)₃ | CH₃ | -CH₂CH₂- | H | 9 |
| 9-B | C(CH3)₃ | CH₂CH₃ | -CH₂CH₂- | H | 9 |
| 9-C | C(CH3)₃ | CH₃ | -CH₂CH₂- | CH₃ | 9 |
| 9-D | C(CH3)₃ | CH₂CH₃ | -CH₂CH₂- | CH₃ | 9 |
| 10-A | C(CH3)₃ | CH₃ | -CH₂CH₂- | H | 10 |
| 10-B | C(CH3)₃ | CH₂CH₃ | -CH₂CH₂- | H | 10 |
| 10-C | C(CH3)₃ | CH₃ | -CH₂CH₂- | CH₃ | 10 |
| 10-D | C(CH3)₃ | CH₂CH₃ | -CH₂CH₂- | CH₃ | 10 |
| 11-A | C(CH3)₃ | CH₃ | -CH₂CH₂- | H | 11 |
| 11-B | C(CH3)₃ | CH₂CH₃ | -CH₂CH₂- | H | 11 |
| 11-C | C(CH3)₃ | CH₃ | -CH₂CH₂- | CH₃ | 11 |
| 11-D | C(CH3)₃ | CH₂CH₃ | -CH₂CH₂- | CH₃ | 11 |
| 12-A | C(CH3)₃ | CH₃ | -CH₂CH₂- | H | 12 |
| 12-B | C(CH3)₃ | CH₂CH₃ | -CH₂CH₂- | H | 12 |
| 12-C | C(CH3)₃ | CH₃ | -CH₂CH₂- | CH₃ | 12 |
| 12-D | C(CH3)₃ | CH₂CH₃ | -CH₂CH₂- | CH₃ | 12 |
| 13-A | C(CH3)₃ | CH₃ | -CH₂CH₂- | H | 13 |
| 13-B | C(CH3)₃ | CH₂CH₃ | -CH₂CH₂- | H | 13 |
| 13-C | C(CH3)₃ | CH₃ | -CH₂CH₂- | CH₃ | 13 |
| 13-D | C(CH3)₃ | CH₂CH₃ | -CH₂CH₂- | CH₃ | 13 |
| 14-A | C(CH3)₃ | CH₃ | -CH₂CH₂- | H | 14 |
| 14-B | C(CH3)₃ | CH₂CH₃ | -CH₂CH₂- | H | 14 |
| 14-C | C(CH3)₃ | CH₃ | -CH₂CH₂- | CH₃ | 14 |
| 14-D | C(CH3)₃ | CH₂CH₃ | -CH₂CH₂- | CH₃ | 14 |
| 15-A | C(CH3)₃ | CH₃ | -CH₂CH₂- | H | 15 |
| 15-B | C(CH3)₃ | CH₂CH₃ | -CH₂CH₂- | H | 15 |
| 15-C | C(CH3)₃ | CH₃ | -CH₂CH₂- | CH₃ | 15 |
| 15-D | C(CH3)₃ | CH₂CH₃ | -CH₂CH₂- | CH₃ | 15 |

Impurities may also result from manufacturing, processing, transportation, storage and use of the inventive materials.

Such impurities can be present from by-products of the reaction process to make the inventive materials, such as loss of an R¹ or R² group, hydrolysis of the ester linkage to a carboxylic acid, single transesterification with the PEG chain resulting in a pegylated ester wherein the PEG terminates in an —OH group. Additional impurities of the mono-ester of PEG impurity can result in autoxidized products, wherein the —OH may form —CHO or —CO₂H groups.

These molecules could be considered as impurities, and their presence will not negate the present invention so long as the majority (and preferably the substantial majority) of the molecules of the composition are as described. In any event, compositions exhibiting this type of variability are to be construed as encompassed by the present invention and the description that a material is “represented by” the formula shown.

Impurities in the phenolic compositions can also result from autoxidation of the product or from trapping of radicals. These impurities are expected to be a part of the product, especially during usage in the detergent or in application. Examples of expected impurities from oxidation can be found in Zhang et al., J Material Sci Eng. 6: 393.

The phenolic compositions disclosed herein may be formulated into a wide variety of laundry care compositions. Suitable laundry care compositions include powdered laundry detergents, detergent tablets and bars, wash additives, laundry detergent liquids including light duty liquids, heavy duty liquids, concentrated liquid detergents, non or low aqueous laundry liquids, unit dose sachets, soap bars, laundry beads, dryer sheets, dissolvable sheets, and combinations thereof. The phenolic composition may be present in an amount from 0.0001 wt. %-1.0 wt. %, from 0.0001 wt. %-0.5 wt. %, from 0.0001 wt. %-0.1 wt. %, from 0.0001 wt. %-0.01 wt. %, from 0.0001 wt. %-0.001 wt. %, from 0.001 wt. %-1.0 wt. %, from 0.001 wt. %-1.0 wt. %, from 0.01 wt. %-1.0 wt. %, from 0.1 wt. %-1.0 wt. %, or from 0.5 wt. %-1.0 wt. %, relative to the total weight of the laundry care composition.

The laundry care composition may be in the form of a liquid composition. The liquid composition may comprise from about 0% to about 99%, or from about 30% to about 90%, or from about 50% to about 80%, by weight of the composition, of water. The liquid composition may include non-aqueous liquid detergents.

The laundry care composition may be in the form of a solid composition. The solid composition may comprise from about 20% to about 98%, by weight of the composition, of a water-soluble carrier for forming solid compositions. In a non-limiting but preferred example, the water-soluble carrier for forming solid compositions may be polyethylene glycol. The polyethylene glycol carrier may have a weight average molecular weight of from about 2000 to about 20,000 Daltons, preferably from about 5000 to about 15,000 Daltons, more preferably from about 6000 to about 12,000 Daltons. The solid composition may comprise less than about 20%, preferably less than about 15%, more preferably less than about 5%, even more preferably less than about 1%, by weight of the composition, of water. In a preferred example, the laundry care composition is in the form of granules or particles. The granules and particles may have a shape selected from the group consisting of spherical, hemispherical, compressed hemispherical, lentil shaped, oblong, and mixtures thereof. One skilled in the art may recognize that these shapes are non-limiting and that the granules and particles may have any other shape known in the art for such granules and particles. The granules may have a maximum dimension (i.e., length, width, height, diameter) of from about 0.1 mm to about 2 mm and a minimum dimension (i.e., length, width, height, diameter) of from about 0.05 mm to about 1.5 mm. The particles may have a maximum dimension (i.e., length, width, height, diameter) of from about 2 mm to about 10 mm and a minimum dimension (i.e., length, width, height, diameter) of from about 1.5 mm to about 4 mm.

The laundry care composition may be free-flowing. Such free-flowing laundry care compositions may be packaged within a container such that a consumer may open the container and simply dose the amount of laundry care composition desired. The container may be any container known in the art suitable for containing laundry care compositions. For example, the container may have a volume of from about 50 cm³ to about 1500 cm³. The container may be of any suitable size and shape for placement on a grocery store shelf, for placement within a consumer's home, or for use within a commercial setting, such as a laundromat.

It is also contemplated that the laundry care composition may be incorporated into a unitized dose article, such as, for example, a single-compartment pouch, a multi-compartment pouch, a dissolvable sheet, a fibrous article, a tablet, a bar, or a mixture thereof. Such pouches typically include a water-soluble film, such as a polyvinyl alcohol water-soluble film, that at least partially encapsulates the laundry care composition. Suitable films include those commercially available from MonoSol, LLC, Indiana, United States. A multi-compartment pouch may comprise at least two, at least three, or at least four compartments. A multi-compartment pouch may include compartments that are side-by-side and/or superposed. The laundry care composition contained in the pouch or compartments thereof may be of liquid form, of solid form, or combinations thereof.

Laundry care compositions encapsulated within pouches may have relatively low amounts of water, for example less than about 20%, or less than about 15%, or less than about 12%, or less than about 10%, or less than about 8%, by weight of the laundry care composition, of water.

The laundry care composition may further include one or more surfactants, for instance in an amount about 0.1% to about 80%, by weight of the laundry care composition, of a surfactant. The surfactant may be selected from the group consisting of nonionic surfactants, anionic surfactants, amphoteric surfactants, zwitterionic surfactants, cationic surfactants, and mixtures thereof. Anionic and nonionic surfactants are typically employed if the laundry care composition is a laundry cleaning composition or detergent. Cationic surfactants are typically employed if the laundry care composition is a fabric softening composition. Surfactants may provide soil removal and assist in dispersing the antioxidant, while not negatively impacting the deposition of the antioxidant onto the fabric.

Suitable nonionic surfactants may include, but are not limited to, alkoxylated fatty alcohols (e.g., ethoxylated fatty alcohols); alkoxylated alkyl phenols; alkyl phenol condensates; mid-chain branched alcohols; mid-chain branched alkyl alkoxylates; alkylpolysaccharides; polyhydroxy fatty acid amides; ether capped poly(oxyalkylated) alcohol surfactants; and mixtures thereof. The alkoxylate units may be ethyleneoxy units, propyleneoxy units, or mixtures thereof. The nonionic surfactant may be linear, branched (e.g., mid-chain branched), or a combination thereof. Examples of suitable nonionic surfactants may include those commercially available under the tradename PLURONIC® from BASF, Ludwigshafen, Germany, those commercially available under the tradename NEODOL® nonionic from Shell, The Hague, The Netherlands, and those commercially available under the tradename SURFONIC® from Huntsman Corporation, The Woodlands, Tex., United States.

Suitable anionic surfactants may include, but are not limited to sulfate detersive surfactants (e.g., alkoxylated and/or non-alkoxylated alkyl sulfate materials); and/or sulfonic detersive surfactants (e.g., alkyl benzene sulfonates). The anionic surfactant may be linear, branched, or combinations thereof. Preferred anionic surfactants may include, but are not limited to, linear alkyl benzene sulfonate (LAS), alkyl ethoxylated sulfate (AES), alkyl sulfates (AS), and mixtures thereof. Other suitable anionic surfactants may include branched modified alkyl benzene sulfonates (MLAS), methyl ester sulfonates (MES), and/or alkyl ethoxylated carboxylates (AEC). The anionic surfactants may be present in acid form, salt form, or mixtures thereof. The anionic surfactant may be neutralized, in part or in whole, for example, by an alkali metal (e.g., sodium) or an amine (e.g., monoethanolamine). The anionic surfactant may be pre-neutralized, preferably with an alkali metal, an alkali earth metal, an amine such as an ethanolamine, or mixtures thereof.

Suitable amphoteric surfactants may include any conventional amphoteric surfactant known to one skilled in the art, such as amine oxides. Preferred amine oxides may include alkyl dimethyl amine oxide or alkyl amido propyl dimethyl amine oxide, more preferably alkyl dimethyl amine oxide and even more preferably coco dimethyl amino oxide. The amine oxide may have a linear or mid-branched alkyl moiety. Typical linear amine oxides may include water-soluble amine oxides containing one C₈₋₁₈alkyl moiety and two moieties selected from the group consisting of C₁₋₃alkyl groups, C₁₋₃hydroxyalkyl groups, and mixtures thereof. Preferably, the amine oxide is characterized by the formula R⁴—N(R⁵)(R⁶)O wherein R⁴ is a C₈₋₁₈ alkyl and R⁵ and R⁶ are selected from the group consisting of methyl, ethyl, propyl, isopropyl, 2-hydroxyethyl, 2-hydroxypropyl and 3-hydroxypropyl. The amine oxide surfactant may include linear C₁₀-C₁₈alkyl dimethyl amine oxides and linear C₈-C₁₂ alkoxy ethyl dihydroxy ethyl amine oxides.

Suitable zwitterionic surfactants may include any conventional zwitterionic surfactant known to one skilled in the art, such as betaines, particularly alkyl betaine, alkylamidobetaine, amidazoliniumbetaine, sulfobetaine, hydroxybetaines, and phosphobetaines. Examples of suitable betaines may include alkyl dimethyl betaine and cocodimethyl amidopropyl betaine, N-alkyl-N,N-dimethylammino-1-propane sulfonate where the alkyl group can be C₈ to C₁₈, or from C₈ to C₁₄. Suitable cationic surfactants may include, but are not limited to, alkyl pyridinium compounds, alkyl quaternary ammonium compounds, alkyl quaternary phosphonium compounds, alkyl ternary sulphonium compounds, and mixtures thereof. Preferred cationic surfactants are quaternary ammonium compounds having the general formula: (R⁷)(R⁸)(R⁹)(R¹⁰—)N⁺X⁻ wherein, R⁷ is a linear or branched, substituted or unsubstituted C₆₋₁₈ alkyl or alkenyl moiety, R⁸ and R⁹ are independently selected from methyl or ethyl moieties, R¹⁰ is a hydroxyl, hydroxymethyl or a hydroxyethyl moiety, X is an anion which provides charge neutrality, preferred anions include: halides, preferably chloride; sulphate; and sulphonate. For the purposes of the present invention, cationic surfactants include those which can deliver fabric care benefits. Non-limiting examples of useful cationic surfactants include: fatty amines, imidazoline quat materials and quaternary ammonium surfactants, preferably N, N-bis(stearoyl-oxy-ethyl) N,N-dimethyl ammonium chloride, N,N-bis(tallowoyl-oxy-ethyl) N,N-dimethyl ammonium chloride, N,N-bis(stearoyl-oxy-ethyl)N-(2 hydroxyethyl)N-methyl ammonium methylsulfate; 1,2-di(stearoyl-oxy) 3-trimethyl ammoniumpropane chloride; dialkylenedimethylammonium salts such as dicanoladimethylammonium chloride, di(hard)tallowdimethylammonium chloride dicanoladimethylammonium methylsulfate; 1-methyl-1-stearoylamidoethyl-2-stearoylimidazolinium methylsulfate; 1-tallowylamidoethyl-2-tallowylimidazoline; N,N″-dialkyldiethylenetriamine; the reaction product of N-(2-hydroxyethyl)-1,2-ethylenediamine or N-(2-hydroxyisopropyl)-1,2-ethylenediamine with glycolic acid, esterified with fatty acid, where the fatty acid is (hydrogenated) tallow fatty acid, palm fatty acid, hydrogenated palm fatty acid, oleic acid, rapeseed fatty acid, hydrogenated rapeseed fatty acid, and a mixture of the above.

The detergent compositions of the present invention may also include any number of additional optional ingredients. These include conventional laundry detergent composition components such as non-tinting dyes, detersive builders, enzymes, enzyme stabilizers (such as propylene glycol, boric acid and/or borax), suds suppressors, soil suspending agents, hueing agents, soil release agents, other fabric care benefit agents, pH adjusting agents, chelating agents, smectite clays, solvents, hydrotropes and phase stabilizers, structuring agents, dye transfer inhibiting agents, opacifying agents, optical brighteners, perfumes and coloring agents. The various optional detergent composition ingredients, if present in the compositions herein, should be utilized at concentrations conventionally employed to bring about their desired contribution to the composition or the laundering operation. Frequently, the total amount of such optional detergent composition ingredients can range from about 0.01% to about 50%, more preferably from about 0.1% to about 30%, by weight of the composition.

Builders—The compositions of the present invention can comprise one or more detergent builders or builder systems. When present, the compositions will typically comprise at least about 1% builder, or from about 5% or 10% to about 80%, 50%, or even 30% by weight, of said builder. Builders include, but are not limited to, the alkali metal, ammonium and alkanolammonium salts of polyphosphates, alkali metal silicates, alkaline earth and alkali metal carbonates, aluminosilicate builders polycarboxylate compounds. ether hydroxypolycarboxylates, copolymers of maleic anhydride with ethylene or vinyl methyl ether, 1,3,5-trihydroxybenzene-2,4,6-trisulphonic acid, and carboxymethyl-oxysuccinic acid, the various alkali metal, ammonium and substituted ammonium salts of polyacetic acids such as ethylenediamine tetraacetic acid and nitrilotriacetic acid, as well as polycarboxylates such as mellitic acid, succinic acid, oxydisuccinic acid, polymaleic acid, benzene 1,3,5-tricarboxylic acid, carboxymethyloxysuccinic acid, and soluble salts thereof.

Chelating Agents—The compositions herein may also optionally contain one or more copper, iron and/or manganese chelating agents. If utilized, chelating agents will generally comprise from about 0.1% by weight of the compositions herein to about 15%, or even from about 3.0% to about 15% by weight of the compositions herein.

Hueing Agents—The compositions of the present invention may also include one or more hueing agents. Suitable hueing agents consist of blue or violet azo colorants, triarylmethane dyes or various leuco dyes. Various non-limiting examples of hueing agents are disclosed in U.S. Pat. Nos. 10,731,112 and 10,876,079. When present in the compositions herein, the hueing dye is present at levels from about 0.0001%, from about 0.01%, from about 0.05% by weight of the cleaning compositions to about 1%, or even about 0.1% by weight of the cleaning compositions.

Dye Transfer Inhibiting Agents—The compositions of the present invention may also include one or more dye transfer inhibiting agents. Suitable polymeric dye transfer inhibiting agents include, but are not limited to, polyvinylpyrrolidone polymers, polyamine N-oxide polymers, copolymers of N-vinylpyrrolidone and N-vinylimidazole, polyvinyloxazolidones and polyvinylimidazoles or mixtures thereof. When present in the compositions herein, the dye transfer inhibiting agents are present at levels from about 0.0001%, from about 0.01%, from about 0.05% by weight of the cleaning compositions to about 10%, about 2%, or even about 1% by weight of the cleaning compositions.

Dispersants—The compositions of the present invention can also contain dispersants. Suitable water-soluble organic materials are the homo- or co-polymeric acids or their salts, in which the polycarboxylic acid may comprise at least two carboxyl radicals separated from each other by not more than two carbon atoms.

Enzymes—The compositions can comprise one or more detergent enzymes which provide cleaning performance and/or fabric care benefits. Examples of suitable enzymes include, but are not limited to, hemicellulases, peroxidases, proteases, cellulases, xylanases, lipases, phospholipases, esterases, cutinases, pectinases, keratanases, reductases, oxidases, phenoloxidases, lipoxygenases, ligninases, pullulanases, tannases, pentosanases, malanases, β-glucanases, arabinosidases, hyaluronidase, chondroitinase, laccase, and amylases, or mixtures thereof. A typical combination is a cocktail of conventional applicable enzymes like protease, lipase, cutinase and/or cellulase in conjunction with amylase.

Enzyme Stabilizers—Enzymes for use in compositions, for example, detergents can be stabilized by various techniques. The enzymes employed herein can be stabilized by the presence of water-soluble sources of calcium and/or magnesium ions in the finished compositions that provide such ions to the enzymes.

Detergent compositions may also contain bleaching agents. Suitable bleaching agents include, for example, hydrogen peroxide sources, such as those described in detail in the herein incorporated Kirk Othmer's Encyclopedia of Chemical Technology, 4th Ed (1992, John Wiley & Sons), Vol. 4, pp. 271-300 “Bleaching Agents (Survey).” These hydrogen peroxide sources include the various forms of sodium perborate and sodium percarbonate, including various coated and modified forms of these compounds.

The preferred source of hydrogen peroxide used herein can be any convenient source, including hydrogen peroxide itself. For example, perborate, e.g., sodium perborate (any hydrate but preferably the mono- or tetra-hydrate), sodium carbonate peroxyhydrate or equivalent percarbonate salts, sodium pyrophosphate peroxyhydrate, urea peroxyhydrate, or sodium peroxide can be used herein. Also useful are sources of available oxygen such as persulfate bleach (e.g., OXONE, manufactured by DuPont). Sodium perborate monohydrate and sodium percarbonate are particularly preferred. Mixtures of any convenient hydrogen peroxide sources can also be used.

Bleach Activators—Preferably, the peroxygen bleach component in the composition is formulated with an activator (peracid precursor). The activator is present at levels of from about 0.01%, preferably from about 0.5%, more preferably from about 1% to about 15%, preferably to about 10%, more preferably to about 8%, by weight of the composition. A bleach activator as used herein is any compound which, when used in conjunction with a hydrogen peroxide, source leads to the in situ production of the peracid corresponding to the bleach activator. Various non-limiting examples of activators are disclosed in U.S. Pat. Nos. 5,576,282; 4,915,854 and 4,412,934. See also U.S. Pat. No. 4,634,551 for other typical bleaches and activators useful herein.

Preferred activators are selected from the group consisting of tetraacetyl ethylene diamine (TAED), benzoylcaprolactam (BzCL), 4-nitrobenzoylcaprolactam, 3-chlorobenzoylcaprolactam, benzoyloxybenzenesulphonate (BOBS), nonanoyloxybenzenesulphonate (NOBS), phenyl benzoate (PhBz), decanoyloxybenzenesulphonate (C₁₀—OBS), benzoylvalerolactam (BZVL), octanoyloxybenzenesulphonate (C₈—OBS), perhydrolyzable esters and mixtures thereof, most preferably benzoylcaprolactam and benzoylvalerolactam. Particularly preferred bleach activators in the pH range from about 8 to about 11 are those selected having an OBS or VL leaving group.

Preferred hydrophobic bleach activators include, but are not limited to, nonanoyloxybenzenesulphonate (NOBS); 4-[N-(nonanoyl) amino hexanoyloxy]-benzene sulfonate sodium salt (NACA-OBS), an example of which is described in U.S. Pat. No. 5,523,434; dodecanoyloxybenzenesulphonate (LOBS or C₁₂—OBS); 10-undecenoyloxybenzenesulfonate (UDOBS or C₁₁—OBS with unsaturation in the 10 position); and decanoyloxybenzoic acid (DOBA).

Preferred bleach activators are those described in U.S. Pat. No. 5,998,350 to Burns et al.; U.S. Pat. No. 5,698,504 to Christie et al.; U.S. Pat. No. 5,695,679 to Christie et al.; U.S. Pat. No. 5,686,401 to Willey et al.; U.S. Pat. No. 5,686,014 to Hartshorn et al.; U.S. Pat. No. 5,405,412 to Willey et al.; U.S. Pat. No. 5,405,413 to Willey et al.; U.S. Pat. No. 5,130,045 to Mitchel et al.; and U.S. Pat. No. 4,412,934 to Chung et al., and copending patent application Ser. No. 08/064,564, all of which are incorporated herein by reference.

Metal-Containing Bleach Catalysts—The compositions and methods of the present invention can also optionally include metal-containing bleach catalysts, preferably manganese and cobalt-containing bleach catalysts.

One type of metal-containing bleach catalyst is a catalyst system comprising a transition metal cation of defined bleach catalytic activity (such as copper, iron, titanium, ruthenium tungsten, molybdenum, or manganese cations), an auxiliary metal cation having little or no bleach catalytic activity (such as zinc or aluminum cations), and a sequestrate having defined stability constants for the catalytic and auxiliary metal cations, particularly ethylenediaminetetraacetic acid, ethylenediaminetetra (methylenephosphonic acid) and water-soluble salts thereof. Such catalysts are disclosed in U.S. Pat. No. 4,430,243 to Bragg.

Bleach Boosting Compounds—The compositions herein may comprise one or more bleach boosting compounds. Bleach boosting compounds provide increased bleaching effectiveness in lower temperature applications. The bleach boosters act in conjunction with conventional peroxygen bleaching sources to provide increased bleaching effectiveness. This is normally accomplished through in situ formation of an active oxygen transfer agent such as a dioxirane, an oxaziridine, or an oxaziridinium. Alternatively, preformed dioxiranes, oxaziridines and oxaziridiniums may be used.

Among suitable bleach boosting compounds for use in accordance with the present invention are cationic imines, zwitterionic imines, anionic imines and/or polyionic imines having a net charge of from about +3 to about −3, and mixtures thereof. These imine bleach boosting compounds of the present invention include those of the general structure:

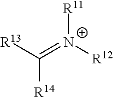

where R¹¹, R¹², R¹³, and R¹⁴ are each independently hydrogen or an unsubstituted or substituted radical selected from the group consisting of phenyl, aryl, heterocyclic ring, alkyl and cycloalkyl radicals.

Among preferred bleach boosting compounds are zwitterionic bleach boosters, which are described in U.S. Pat. Nos. 5,576,282 and 5,718,614. Other bleach boosting compounds include cationic bleach boosters described in U.S. Pat. Nos. 5,360,569; 5,442,066; 5,478,357; 5,370,826; 5,482,515; 5,550,256; and WO 95/13351, WO 95/13352, and WO 95/13353.

Peroxygen sources are well-known in the art and the peroxygen source employed in the present invention may comprise any of these well known sources, including peroxygen compounds as well as compounds, which under consumer use conditions, provide an effective amount of peroxygen in situ. The peroxygen source may include a hydrogen peroxide source, the in situ formation of a peracid anion through the reaction of a hydrogen peroxide source and a bleach activator, preformed peracid compounds or mixtures of suitable peroxygen sources. Of course, one of ordinary skill in the art will recognize that other sources of peroxygen may be employed without departing from the scope of the invention. The bleach boosting compounds, when present, are preferably employed in conjunction with a peroxygen source in the bleaching systems of the present invention.

Photobleaches—Suitable photobleaches for use in the treating compositions of the present invention include, but are not limited to, the photobleaches described in U.S. Pat. Nos. 4,217,105 and 5,916,481.

Enzyme Bleaching—Enzymatic systems may be used as bleaching agents. The hydrogen peroxide may also be present by adding an enzymatic system (i.e. an enzyme and a substrate therefore) which is capable of generating hydrogen peroxide at the beginning or during the washing and/or rinsing process. Such enzymatic systems are disclosed in EP Patent Application 91202655.6 filed Oct. 9, 1991.

The liquid detergent compositions are in the form of an aqueous solution or uniform dispersion or suspension of surfactant, phenolic composition, and certain optional other ingredients, some of which may normally be in solid form, that have been combined with the normally liquid components of the composition, such as the liquid alcohol ethoxylate nonionic, the aqueous liquid carrier, and any other normally liquid optional ingredients. Such a solution, dispersion or suspension will be acceptably phase stable and will typically have a viscosity which ranges from about 100 to 600 cps, more preferably from about 150 to 400 cps. For purposes of this invention, viscosity is measured with a Brookfield LVDV-II+ viscometer apparatus using a #21 spindle.

The liquid detergent compositions herein can be prepared by combining the components thereof in any convenient order and by mixing, e.g., agitating, the resulting component combination to form a phase stable liquid detergent composition. In a preferred process for preparing such compositions, a liquid matrix is formed containing at least a major proportion, and preferably substantially all, of the liquid components, e.g., nonionic surfactant, the non-surface active liquid carriers and other optional liquid components, with the liquid components being thoroughly admixed by imparting shear agitation to this liquid combination. For example, rapid stirring with a mechanical stirrer may usefully be employed. While shear agitation is maintained, substantially all of any anionic surfactants and the solid form ingredients can be added. Agitation of the mixture is continued, and if necessary, can be increased at this point to form a solution or a uniform dispersion of insoluble solid phase particulates within the liquid phase. After some or all of the solid-form materials have been added to this agitated mixture, particles of any enzyme material to be included, e.g., enzyme prills, are incorporated. As a variation of the composition preparation procedure hereinbefore described, one or more of the solid components may be added to the agitated mixture as a solution or slurry of particles premixed with a minor portion of one or more of the liquid components. After addition of all of the composition components, agitation of the mixture is continued for a period of time sufficient to form compositions having the requisite viscosity and phase stability characteristics. Frequently this will involve agitation for a period of from about 30 to 60 minutes.

In an alternate embodiment for forming the liquid detergent compositions, the phenolic composition is first combined with one or more liquid components to form a phenolic composition premix, and this premix is added to a composition formulation containing a substantial portion, for example more than 50% by weight, more specifically, more than 70% by weight, and yet more specifically, more than 90% by weight, of the balance of components of the laundry detergent composition. For example, in the methodology described above, both the phenolic composition premix and the enzyme component are added at a final stage of component additions. In a further embodiment, the phenolic composition is encapsulated prior to addition to the detergent composition, the encapsulated phenolic composition is suspended in a structured liquid, and the suspension is added to a composition formulation containing a substantial portion of the balance of components of the laundry detergent composition.

The compositions of this invention, prepared as hereinbefore described, can be used to form aqueous washing solutions for use in the laundering of textile substrates such as fabrics. Generally, an effective amount of such compositions is added to water, preferably in a conventional fabric laundering automatic washing machine, to form such aqueous laundering solutions. The aqueous washing solution so formed is then contacted, preferably under agitation, with the fabrics to be laundered therewith. An effective amount of the liquid detergent compositions described herein is added to water to form aqueous laundering solutions comprising from about 500 to 7,000 ppm of the detergent composition in the aqueous washing solution. More preferably, from about 1,000 to 3,000 ppm of the detergent compositions described herein will be provided in aqueous washing solution.

### Fabric Treatment Compositions/Rinse Added Fabric Softening Compositions

In another specific embodiment, the phenolic composition of the present invention may be included in a fabric treatment composition. The fabric treatment composition may be comprised of at least one phenolic composition and a rinse added fabric softening composition (“RAFS;” also known as rinse added fabric conditioning compositions). Examples of typical rinse added softening compositions can be found in U.S. Provisional Patent Application Ser. No. 60/687,582 filed on Oct. 8, 2004. The rinse added fabric softening compositions of the present invention may comprise (a) fabric softening active (“FSA”) and (b) phenolic composition. The rinse added fabric softening composition may comprise from about 1% to about 90% by weight of the FSA, more preferably from about 5% to about 50% by weight of the FSA. The phenolic composition may be present in the rinse added fabric softening composition in an amount from about 0.5 ppb to about 10000 ppm, more preferably from about 0.5 ppm to about 1000 ppm.

In one embodiment of the invention, the fabric softening active is a quaternary ammonium compound suitable for softening fabric in a rinse step. In one embodiment, the FSA is formed from a reaction product of a fatty acid and an aminoalcohol obtaining mixtures of mono-, di-, and, in one embodiment, triester compounds. In another embodiment, the FSA comprises one or more softener quaternary ammonium compounds such, but not limited to, as a monoalkyquaternary ammonium compound, a diamido quaternary compound and a diester quaternary ammonium compound, or a combination thereof.

In one aspect of the invention, the FSA comprises a diester quaternary ammonium (hereinafter “DQA”) compound composition. In certain embodiments of the present invention, the DQA compounds compositions also encompasses a description of diamido FSAs and FSAs with mixed amido and ester linkages as well as the aforementioned diester linkages, all herein referred to as DQA.

A first type of DQA (“DQA (1)”) suitable as a FSA includes a compound comprising the formula:

{R¹⁵₄₋ₘ—N⁺—[(CH₂)ₙ—Y—R¹⁶]ₘ}X⁻

wherein each R¹⁵ substituent is either hydrogen, a short chain C₁-C₆, preferably C₁-C₃ alkyl or hydroxyalkyl group, e.g., methyl (most preferred), ethyl, propyl, hydroxyethyl, and the like, poly (C₂₋₃ alkoxy), preferably polyethoxy, group, benzyl, or mixtures thereof; each m is 2 or 3; each n is from 1 to about 4, preferably 2; each Y is —O—(O)C—, —C(O)—O—, —NR—C(O)—, or —C(O)—NR— and it is acceptable for each Y to be the same or different; the sum of carbons in each R¹, plus one when Y is —O—(O)C— or —NR—C(O)—, is C₁₂-C₂₂, preferably C₁₄-C₂₀, with each R¹⁶ being a hydrocarbyl, or substituted hydrocarbyl group; it is acceptable for R¹ to be unsaturated or saturated and branched or linear and preferably it is linear; it is acceptable for each R¹ to be the same or different and preferably these are the same; and X⁻ can be any softener-compatible anion, preferably, chloride, bromide, methylsulfate, ethylsulfate, sulfate, phosphate, and nitrate, more preferably chloride or methyl sulfate. Preferred DQA compounds are typically made by reacting alkanolamines such as MDEA (methyldiethanolamine) and TEA (triethanolamine) with fatty acids. Some materials that typically result from such reactions include N,N-di(acyl-oxyethyl)-N,N-dimethylammonium chloride or N,N-di(acyl-oxyethyl)-N,N-methylhydroxyethylammonium methylsulfate wherein the acyl group is derived from animal fats, unsaturated, and polyunsaturated, fatty acids, e.g., tallow, hardened tallow, oleic acid, and/or partially hydrogenated fatty acids, derived from vegetable oils and/or partially hydrogenated vegetable oils, such as, canola oil, safflower oil, peanut oil, sunflower oil, corn oil, soybean oil, tall oil, rice bran oil, palm oil, etc.

Non-limiting examples of suitable fatty acids are listed in U.S. Pat. No. 5,759,990 at column 4, lines 45-66. In one embodiment, the FSA comprises other actives in addition to DQA (1) or DQA. In yet another embodiment, the FSA comprises only DQA (1) or DQA and is free or essentially free of any other quaternary ammonium compounds or other actives. In yet another embodiment, the FSA comprises the precursor amine that is used to produce the DQA.

In another aspect of the invention, the FSA comprises a compound, identified as DTTMAC comprising the formula:

[R¹⁷₄₋ₘ—N⁽⁺⁾—R¹⁸ₘ]A⁻

wherein each m is 2 or 3, each R¹⁸ is a C₆-C₂₂, preferably C₁₄-C₂₀, but no more than one being less than about C₁₂ and then the other is at least about 16, hydrocarbyl, or substituted hydrocarbyl substituent, preferably C₁₀-C₂₀ alkyl or alkenyl (unsaturated alkyl, including polyunsaturated alkyl, also referred to sometimes as “alkylene”), most preferably C₁₂-C₁₈ alkyl or alkenyl, and branch or unbranched. In one embodiment, the Iodine Value (IV) of the FSA is from about 1 to 70; each R¹⁷ is H or a short chain C₁-C₆, preferably C₁-C₃ alkyl or hydroxyalkyl group, e.g., methyl (most preferred), ethyl, propyl, hydroxyethyl, and the like, benzyl, or (R¹⁹O)₂₋₄H where each R¹⁹ is a C₁₋₆ alkylene group; and A⁻ is a softener compatible anion, preferably, chloride, bromide, methylsulfate, ethylsulfate, sulfate, phosphate, or nitrate; more preferably chloride or methyl sulfate.

Examples of these FSAs include dialkydimethylammonium salts and dialkylenedimethylammonium salts such as ditallowdimethylammonium and ditallowdimethylammonium methylsulfate. Examples of commercially available dialkylenedimethylammonium salts usable in the present invention are di-hydrogenated tallow dimethyl ammonium chloride and ditallowdimethyl ammonium chloride available from Degussa under the trade names Adogen® 442 and Adogen® 470 respectively. In one embodiment, the FSA comprises other actives in addition to DTTMAC. In yet another embodiment, the FSA comprises only compounds of the DTTMAC and is free or essentially free of any other quaternary ammonium compounds or other actives.

In one embodiment, the FSA comprises an FSA described in U.S. Pat. Pub. No. 2004/0204337 A1, published Oct. 14, 2004 to Corona et al., from paragraphs 30-79. In another embodiment, the FSA is one described in U.S. Pat. Pub. No. 2004/0229769 A1, published Nov. 18, 2005, to Smith et al., on paragraphs 26-31; or U.S. Pat. No. 6,494,920, at column 1, line 51 et seq. detailing an “esterquat” or a quaternized fatty acid triethanolamine ester salt.

In one embodiment, the FSA is chosen from at least one of the following: ditallowoyloxyethyl dimethyl ammonium chloride, dihydrogenated-tallowoyloxyethyl dimethyl ammonium chloride, ditallow dimethyl ammonium chloride, ditallowoyloxyethyl dimethyl ammonium methyl sulfate, dihydrogenated-tallowoyloxyethyl dimethyl ammonium chloride, dihydrogenated-tallowoyloxyethyl dimethyl ammonium chloride, or combinations thereof.

In one embodiment, the FSA may also include amide containing compound compositions. Examples of diamide comprising compounds may include but not limited to methyl-bis(tallowamidoethyl)-2-hydroxyethylammonium methyl sulfate (available from Degussa under the trade names Varisoft 110 and Varisoft 222). An example of an amide-ester containing compound is N-[3-(stearoylamino)propyl]-N-[2-(stearoyloxy)ethoxy)ethyl)]-N-methylamine.

Another specific embodiment of the invention provides for a rinse added fabric softening composition further comprising a cationic starch. Cationic starches are disclosed in US 2004/0204337 A1. In one embodiment, the rinse added fabric softening composition comprises from about 0.1% to about 7% of cationic starch by weight of the fabric softening composition. In one embodiment, the cationic starch is HCP401 from National Starch.

### Exemplary Laundry Care Composition Formulations:

### Liquid Detergent Formulations:

Table A provides examples of liquid detergent formulations which include at least one phenolic composition of the present invention.

TABLE A
Liquid Detergent Formulations Comprising Phenolic Composition

| | 1a | 1b | 1c | 1d | 1e | 1f ⁴ |
|---|---|---|---|---|---|---|
| Ingredient | wt % | wt % | wt % | wt % | wt % | wt % |
| sodium alkyl ether sulfate | 14.4% | 14.4% | | 9.2% | 5.4% | |
| linear alkylbenzene sulfonic | 4.4% | 4.4% | 12.2% | 5.7% | 1.3% | 22.0% |
| acid | | | | | | |
| alkyl ethoxylate | 2.2% | 2.2% | 8.8% | 8.1% | 3.4% | 18.0% |
| amine oxide | 0.7% | 0.7% | 1.5% | | | |
| citric acid | 2.0% | 2.0% | 3.4% | 1.9% | 1.0% | 1.6% |
| fatty acid | 3.0% | 3.0% | 8.3% | | | 16.0% |
| Protease | 1.0% | 1.0% | 0.7% | 1.0% | | 2.5% |
| Amylase | 0.2% | 0.2% | 0.2% | | | 0.3% |
| Lipase | | | | 0.2% | | |
| Borax | 1.5% | 1.5% | 2.4% | 2.9% | | |
| calcium and sodium formate | 0.2% | 0.2% | | | | |
| formic acid | | | | | | 1.1% |
| amine ethoxylate polymers | 1.8% | 1.8% | 2.1% | | | 3.2% |
| sodium polyacrylate | | | | | 0.2% | |
| sodium polyacrylate copolymer | | | | 0.6% | | |
| DTPA¹ | 0.1% | 0.1% | | | | 0.9% |
| DTPMP² | | | 0.3% | | | |
| EDTA³ | | | | | 0.1% | |
| fluorescent whitening agent | 0.15% | 0.15% | 0.2% | 0.12% | 0.12% | 0.2% |
| Ethanol | 2.5% | 2.5% | 1.4% | 1.5% | | |
| propanediol | 6.6% | 6.6% | 4.9% | 4.0% | | 15.7% |
| Sorbitol | | | | 4.0% | | |
| ethanolamine | 1.5% | 1.5% | 0.8% | 0.1% | | 11.0% |
| sodium hydroxide | 3.0% | 3.0% | 4.9% | 1.9% | 1.0% | |
| sodium cumene sulfonate | | | 2.0% | | | |
| silicone suds suppressor | | | 0.01% | | | |
| Perfume | 0.3% | 0.3% | 0.7% | 0.3% | 0.4% | 0.6% |
| Phenolic Composition | 0.013% | 0.001% | 0.1% | 1% | 0.0005% | 0.001% |
| Water | balance | Balance | balance | balance | balance | Balance |
| | 100.0% | 100.0% | 100.0% | 100.0% | 100.0% | 100.0% |

¹diethylenetriaminepentaacetic acid, sodium salt
²diethylenetriaminepentakismethylenephosphonic acid, sodium salt
³ethylenediaminetetraacetic acid, sodium salt
⁴ a compact formula, packaged as a unitized dose in polyvinyl alcohol film

### Granular Detergent Formulations:

Table B provides examples of granular detergent formulations which include at least one phenolic composition of the present invention.

TABLE B
Granular Detergent Formulations Comprising Phenolic Composition

| Ingredient | 2a | 2b | 2c | 2d | 2e |
|---|---|---|---|---|---|
| | wt % | wt % | wt % | wt % | wt % |
| Na linear alkylbenzene sulfonate | 3.4% | 3.3% | 11.0% | 3.4% | 3.3% |
| Na alkylsulfate | 4.0% | 4.1% | | 4.0% | 4.1% |
| Na alkyl sulfate (branched) | 9.4% | 9.6% | | 9.4% | 9.6% |
| alkyl ethoxylate | | | 3.5% | | |
| type A zeolite | 37.4% | 35.4% | 26.8% | 37.4% | 35.4% |
| sodium carbonate | 22.3% | 22.5% | 35.9% | 22.3% | 22.5% |
| sodium sulfate | 1.0% | | 18.8% | 1.0% | |
| sodium silicate | | | 2.2% | | |
| Protease | 0.1% | 0.2% | | 0.1% | 0.2% |
| sodium polyacrylate | 1.0% | 1.2% | 0.7% | 1.0% | 1.2% |
| carboxymethylcellulose | | | 0.1% | | |
| PEG 600 | | 0.5% | | | 0.5% |
| PEG 4000 | | 2.2% | | | 2.2% |
| DTPA | 0.7% | 0.6% | | 0.7% | 0.6% |
| fluorescent whitening agent | 0.1% | 0.1% | 0.1% | 0.1% | 0.1% |
| sodium percarbonate | | 5.0% | | | 5.0% |
| sodium nonanoyloxybenzenesulfonate | | 5.3% | | | 5.3% |
| silicone suds suppressor | 0.02% | 0.02% | | 0.02% | 0.02% |
| Perfume | 0.3% | 0.3% | 0.2% | 0.3% | 0.3% |
| Phenolic composition | 0.004% | 0.1% | 0.04% | 1% | 0.02% |
| water and miscellaneous | balance | balance | balance | balance | Balance |
| | 100.0% | 100.0% | 100.0% | 100.0% | 100.0% |

### Fabric Treatment Compositions:

Table C provides examples of liquid fabric treatment compositions which include at least one phenolic composition of the present invention.

TABLE C
Liquid Fabric Treatment Compositions Comprising Phenolic Composition

| Ingredients | a | b | c | d |
|---|---|---|---|---|
| Fabric Softening Active ^{a} | 13.70% | 13.70% | 13.70% | 13.70% |
| Ethanol | 2.14% | 2.14% | 2.14% | 2.14% |
| Cationic Starch ^{b} | 2.17% | 2.17% | 2.17% | 2.17% |
| Perfume | 1.45% | 1.45% | 1.45% | 1.45% |
| Phase Stabilizing Polymer ^{c} | 0.21% | 0.21% | 0.21% | 0.21% |
| Calcium Chloride | 0.147% | 0.147% | 0.147% | 0.147% |
| DTPA^{d} | 0.007% | 0.007% | 0.007% | 0.007% |
| Preservative ^{e} | 5 ppm | 5 ppm | 5 ppm | 5 ppm |
| Antifoam ^{f} | 0.015% | 0.015% | 0.015% | 0.015% |
| Phenolic Composition | 0.1% | 0.01% | 1% | 0.0001% |
| Tinopal CBS-X ^{g} | 0.2 | 0.2 | 0.2 | 0.2 |
| Ethoquad C/25 ^{h} | 0.26 | 0.26 | 0.26 | 0.26 |
| Ammonium Chloride | 0.1% | 0.1% | 0.1% | 0.1% |
| Hydrochloric Acid | 0.012% | 0.012% | 0.012% | 0.012% |
| Deionized Water | Balance | Balance | Balance | Balance |

^{a} N,N-di(tallowoyloxyethyl)-N,N-dimethylammonium chloride.
^{b} Cationic starch based on common maize starch or potato starch, containing 25% to 95% amylose and a degree of substitution of from 0.02 to 0.09, and having a viscosity measured as Water Fluidity having a value from 50 to 84.
^{c} Copolymer of ethylene oxide and terephthalate having the formula described in US 5,574,179 at col. 15, lines 1-5, wherein each X is methyl, each n is 40, u is 4, each R¹ is essentially 1,4-phenylene moieties, each R² is essentially ethylene, 1,2-propylene moieties, or mixtures thereof.
^{d} Diethylenetriaminepentaacetic acid.
^{e} KATHON ® CG available from Rohm and Haas Co.
^{f} Silicone antifoam agent available from Dow Corning Corp. under the trade name DC2310.
^{g} Disodium 4,4’-bis-(2-sulfostyryl) biphenyl, available from Ciba Specialty Chemicals.
^{h} Cocomethyl ethoxylated [15] ammonium chloride, available from Akzo Nobel.

Textile substrates treated with the laundry care composition(s) comprised of the phenolic composition of the present invention may be comprised of synthetic fibers, natural fibers, or combinations of synthetic and natural fibers. Synthetic fibers include, for example, polyester, acrylic, polyamide, polyolefin, polyaramid, polyurethane, regenerated cellulose (i.e., rayon), and blends thereof. The term “polyamide” is intended to describe any long-chain polymer having recurring amide groups (—NH—CO—) as an integral part of the polymer chain. Examples of polyamides include nylon 6; nylon 6, 6; nylon 1, 1; and nylon 6, 10. The term “polyester” is intended to describe any long-chain polymer having recurring ester groups (—C(O)—O—). Examples of polyesters include aromatic polyesters, such as polyethylene terephthalate (PET), polybutylene terephthalate (PBT), polytrimethylene terephthalate (PTT), and polytriphenylene terephthalate, and aliphatic polyesters, such as polylactic acid (PLA). “Polyolefin” includes, for example, polypropylene, polyethylene, and combinations thereof. “Polyaramid” includes, for example, poly-p-phenyleneteraphthalamid (i.e., Kevlar®), poly-m-phenyleneteraphthalamid (i.e., Nomex®), and combinations thereof. Natural fibers include, for example, wool, cotton, flax, and blends thereof.

The textile substrate may be of any variety, including but not limited to, woven fabric, knitted fabric, nonwoven fabric, or combinations thereof. The textile substrate may optionally be colored by a variety of dyeing techniques, such as high temperature jet dyeing with disperse dyes, vat dyeing, thermosol dyeing, pad dyeing, transfer printing, screen printing, or any other technique that is common in the art for comparable textile products. The yarns or fibers comprising the textile substrate may optionally be dyed by suitable methods prior to fabric formation, such as, for instance, by package dyeing or solution dyeing.

Textile substrates include, for example, articles of apparel, such as outerwear (e.g., rainwear), workwear (e.g., uniforms), fashion apparel (e.g., shirts, pants, and other garments); drapery; napery (e.g., table linens and napkins); residential upholstery; commercial upholstery; automotive upholstery; wall coverings; floorcovering articles (e.g., carpets, rugs and mats); human bedding (e.g., mattresses, mattress covers, and the like); pet bedding; outdoor fabric (e.g., outdoor furniture, awnings, boat covers, and grill covers); medical dressings (e.g., fabrics for use in wound care); and any other article capable of possessing discoloration and wherein it is desirable to control (e.g. prevent, remove, and/or improve) said discoloration.

### Description of Certain Specific Embodiments

The following listing describes certain specific embodiments of the phenolic compositions and laundry care compositions described above.

1. A laundry care composition comprising:
a) a surfactant; and
b) a phenolic composition comprising:
   i) a first phenolic compound having the formula:

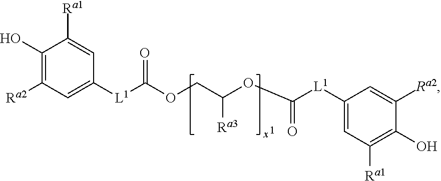

wherein L¹ is selected from null, —CH₂—, —CH₂CH₂—, and —CH₂CH₂CH₂—,
x¹ is an integer from 2-50;
R^{a1} is a C₁₋₈ alkyl group;
R^{a2} is a C₁₋₈ alkyl group;
R^{a3} is H, CH₃, or CH₂CH₃;
ii) a second phenolic compound having the formula:

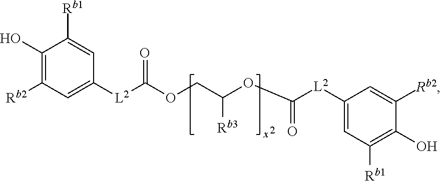

wherein L² is selected from null, —CH₂—, —CH₂CH₂—, and —CH₂CH₂CH₂—,
x² is an integer from 2-50; provided that x¹ and x² are not the same;
R^{b1} is a C₁₋₈ alkyl group;
R^{b2} is a C₁₋₈ alkyl group; and
R^{b3} is H, CH₃, or CH₂CH₃.
2. The laundry care composition according to any preceding embodiment, wherein the phenolic composition is present in an amount from 0.0001 wt. %-1.0 wt. %, from 0.0001 wt. %-0.5 wt. %, from 0.0001 wt. %-0.1 wt. %, from 0.0001 wt. %-0.01 wt. %, from 0.0001 wt. %-0.001 wt. %, from 0.001 wt. %-1.0 wt. %, from 0.001 wt. %-1.0 wt. %, from 0.01 wt. %-1.0 wt. %, from 0.1 wt. %-1.0 wt. %, or from 0.5 wt. %-1.0 wt. %, relative to the total weight of the laundry care composition.
3. The laundry care composition according to any preceding embodiment, wherein R^{a1} and R^{a2} are not the same.
4. The laundry care composition according to any preceding embodiment, wherein R^{b1} and R^{a2} are not the same.
5. The laundry care composition according to any preceding embodiment, wherein R^{a2} and R^{b2} are each C₃₋₅ alkyl.
6. The laundry care composition according to any preceding embodiment, wherein R^{a2} and R^{b2} are t-butyl.
7. The laundry care composition according to any preceding embodiment, wherein R^{a1} and R^{b1} are each a linear C₁₋₈ alkyl group.
8. The laundry care composition according to any preceding embodiment, wherein R^{a1} and R^{b1} are each a linear C₁₋₄ alkyl group.
9. The laundry care composition according to any preceding embodiment, wherein R^{a1} and R^{b1} are each methyl, or R^{a1} and R^{b1} are each ethyl.
10. The laundry care composition according to any preceding embodiment, wherein L¹ is —CH₂CH₂— or —CH₂CH₂CH₂—.
11. The laundry care composition according to any preceding embodiment, wherein L² is —CH₂CH₂— or —CH₂CH₂CH₂—.
12. The laundry care composition according to any preceding embodiment, wherein x¹ is an integer from 4-25, from 4-20, from 4-15, from 4-10, from 4-8, or from 4-6.
13. The laundry care composition according to any preceding embodiment, wherein x² is an integer from 2-25, from 2-20, from 2-15, from 2-10, from 2-8, from 2-6, or from 2-4.
14. The laundry care composition according to any preceding claim, wherein R^{a3} and R^{b3} are each H.
15. The laundry care composition according to any preceding embodiment, wherein the molar ratio of the first phenolic compound to the second phenolic compound is from 50:1 to 1:50, from 25:1 to 1:25, from 20:1 to 1:20, from 10:1 to 1:10, from 5:1 to 1:5, or from 2:1 to 1:2.
16. The laundry care composition according to any preceding embodiment, further comprising a third phenolic compound having the formula:

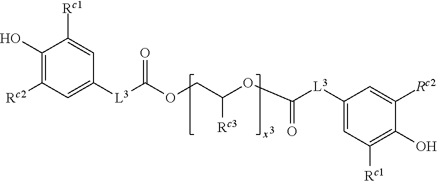

wherein L³ is selected from null, —CH₂—, —CH₂CH₂—, and —CH₂CH₂CH₂—,
x³ is an integer from 2-50; provided that none of x¹, x², and x³ are the same;
R^{c1} is a C₁₋₈ alkyl group;
R^{c2} is a C₁₋₈ alkyl group; and
R^{c3} is H, CH₃, or CH₂CH₃.
17. The laundry care composition according to any preceding embodiment, further comprising a fourth phenolic compound having the formula:

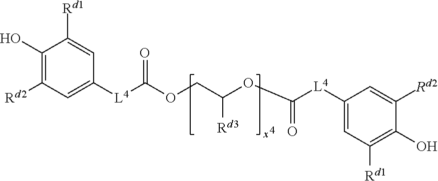

wherein L⁴ is selected from null, —CH₂—, —CH₂CH₂—, and —CH₂CH₂CH₂—,
x⁴ is an integer from 2-50; provided that none of x¹, x², x³, and x⁴ are the same;
R^{d1} is a C₁₋₈ alkyl group;
R^{d2} is a C₁₋₈ alkyl group; and
R^{d3} is H, CH₃, or CH₂CH₃.
18. The laundry care composition according to any preceding embodiment, further comprising a fifth phenolic compound having the formula:

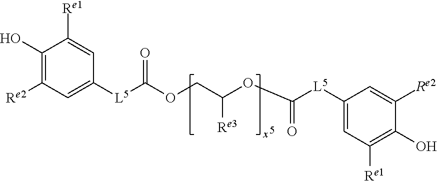

wherein L⁵ is selected from null, —CH₂—, —CH₂CH₂—, and —CH₂CH₂CH₂—,
x⁵ is an integer from 2-50; provided that none of x¹, x², x³, x⁴, and x⁵ are the same;
R^{e1} is a C₁₋₈ alkyl group;
R^{e2} is a C₁₋₈ alkyl group; and
R^{e3} is H, CH₃, or CH₂CH₃.
19. The laundry care composition according to any preceding embodiment, further comprising a sixth phenolic compound having the formula:

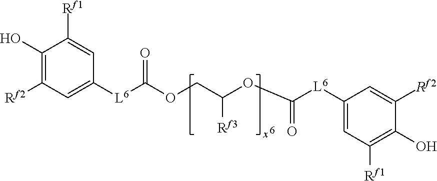

wherein L⁶ is selected from null, —CH₂—, —CH₂CH₂—, and —CH₂CH₂CH₂—,
x⁶ is an integer from 2-50; provided that none of x¹, x², x³, x⁴, x⁵, and x⁶ are the same;
R^{f1} is a C₁₋₈ alkyl group;
R^{f2} is a C₁₋₈ alkyl group; and
R^{f3} is H, CH₃, or CH₂CH₃.
20. The laundry care composition according to any preceding embodiment, further comprising a seventh phenolic compound having the formula:

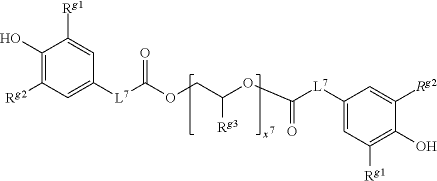

wherein L⁷ is selected from null, —CH₂—, —CH₂CH₂—, and —CH₂CH₂CH₂—,
x⁷ is an integer from 2-50; provided that none of x¹, x², x³, x⁴, x⁵, x⁶, and x⁷ are the same;
R^{g1} is a C₁₋₈ alkyl group;
R^{g2} is a C₁₋₈ alkyl group; and
R^{g3} is H, CH₃, or CH₂CH₃.
21. The laundry care composition according to any preceding embodiment, further comprising an eighth phenolic compound having the formula:

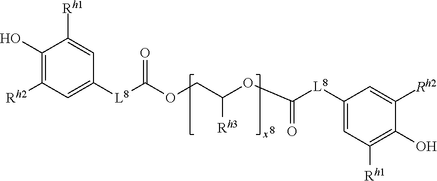

wherein L⁸ is selected from null, —CH₂—, —CH₂CH₂—, and —CH₂CH₂CH₂—,
x⁸ is an integer from 2-50; provided that none of x¹, x², x³, x⁴, x⁵, x⁶, x⁷, and x⁸ are the same;
R^{h1} is a C₁₋₈ alkyl group;
R^{h2} is a C₁₋₈ alkyl group; and
R^{h3} is H, CH₃, or CH₂CH₃.
22. The laundry care composition according to any preceding embodiment, wherein the composition is selected from the group consisting of powdered laundry detergents, detergent tablets and bars, wash additives, laundry detergent liquids including light duty liquids, heavy duty liquids, concentrated liquid detergents, non or low aqueous laundry liquids, unit dose sachets, soap bars . . . .
23. The laundry care composition according to any preceding embodiment, wherein the composition is a liquid and comprises one or more solvents selected from water, C₃₋₈ monoalcohols, C₃₋₈ polyalcohols . . .
24. The laundry care composition according to any preceding embodiment, comprising an alkali metal salt, an alkaline earth metal salt or a salt of an inorganic or C₁₋₆alkyl organic acid, or a mixture thereof.
25. The laundry care composition according to any preceding embodiment, wherein the laundry care composition does not include an oligoamine.
26. A method of laundering an article comprising contacting the article with the composition according to any preceding embodiment.
27. The method according to any preceding embodiment, wherein the article is contacted with the composition in the presence of water.
28. The method according to any preceding embodiment, where the article is contacted with the composition in the presence of water at a temperature no greater than 40° C., no greater than 35° C., no greater than 30° C., no greater than 25° C., no greater than 20° C., no greater than 15° C. no greater than 10° C., or no greater than 5° C.
29. The method according to any preceding embodiment, wherein the article is contacted with the composition is the presence of water at room temperature or less.
30. A method of manufacturing a laundry care composition, comprising combining a water-soluble carrier, a surfactant and a phenolic composition, said phenolic composition comprising:
   a first phenolic compound having the formula:

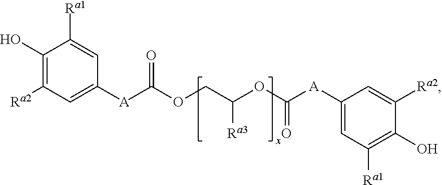

wherein A is selected from null, —CH₂—, —CH₂CH₂—, and —CH₂CH₂CH₂—,
x is an integer from 2-50;
R^{a1} is a C₁₋₈ alkyl group;
R^{a2} is a C₁₋₈ alkyl group;
R^{a3} is H, CH₃, or CH₂CH₃; and
a second phenolic compound having the formula:

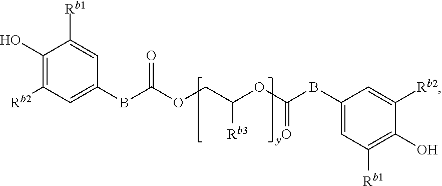

wherein B is selected from null, —CH₂—, —CH₂CH₂—, and —CH₂CH₂CH₂—,
y is an integer from 2-50; provided that x and y are not the same;
R^{b1} is C₁₋₈ alkyl group;
R^{b2} is a C₁— alkyl group; and
R^{b3} is H, CH₃, or CH₂CH₃;
31. A laundry care composition obtained by the process of any preceding embodiment.

### EXAMPLES

The following examples are for the purpose of illustration of the invention only and are not intended to limit the scope of the present invention in any manner whatsoever.

### Example 1: Synthesis of Phenolic Compositions

### Compound 1: Methyl 3-[3-(tert-butyl)-4-hydroxy-5-tolyl]propionate

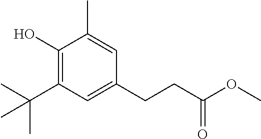 [SMILES: COC(=O)CCc1cc(C)c(O)c(C(C)(C)C)c1]

A 1000 mL 4-neck round bottom flask was affixed with temperature probe, overhead stirring and a reflux condenser under a nitrogen atmosphere. To the reaction flask was charged, 100 g 2-[2-(2-{3-[3-(tert-Butyl)-4-hydroxy-5-tolyl]propionoxy}ethoxy)ethoxy]ethyl 3-[3-(tert-butyl)-4-hydroxy-5-tolyl]propionate (Irganox 245 available from BASF), 6 g of a 30% sodium methoxide solution in methanol and 200 g of methanol. The reaction was stirred at 20-25° C. for 16 hours and then neutralized with 2 g of acetic acid. To the reaction flask was added 100 mL of distilled water and 100 mL of hexane, then the contents of the flask were transferred to a separatory funnel followed by an additional 300 mL of distilled water. The phases were separated and the organic layer was collected and dried under reduced pressure to obtain methyl 3-[3-(tert-butyl)-4-hydroxy-5-tolyl]propionate as an oil that solidified upon standing.

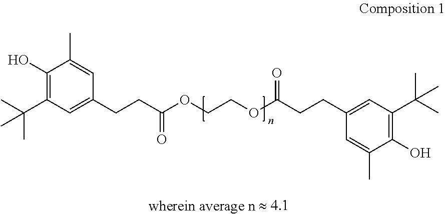

A 100 mL 3-neck round bottom flask was affixed with a temperature probe, overhead stirring and a dean-stark trap with a condenser under a nitrogen atmosphere. To the reaction flask was charged, 20 g of methyl 3-[3-(tert-butyl)-4-hydroxy-5-tolyl]propionate, 8 g of PEG 200 and 0.4 g dibutyltin oxide. The reaction was heated to 240° C. and stirred until it was judged complete by TLC analysis (about 18-24 hours). The product was obtained as a brown oil. The product was a composition containing a distribution of phenolic compounds encompassed by the formula above, and the value of n is equal to the average number of ethylene oxide repeat units of the phenolic compounds present in the composition.

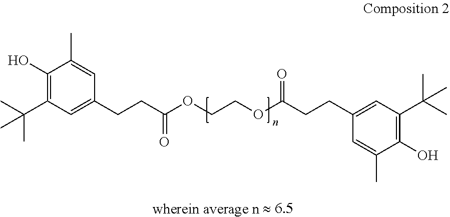

The same procedure as in Composition 1, except 12 g of PEG 300 was used in place of the PEG 200. The product was obtained as a dark yellow oil. The product was a composition containing a distribution of phenolic compounds encompassed by the formula above, and the value of n is equal to the average number of ethylene oxide repeat units of the phenolic compounds present in the composition.

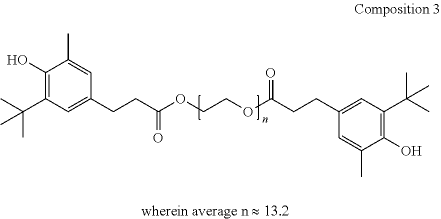

The same procedure as in Composition 1, except 24 g of PEG 600 was used in place of the PEG 200. The product was obtained as a dark yellow oil. The product was a composition containing a distribution of phenolic compounds encompassed by the formula above, and the value of n is equal to the average number of ethylene oxide repeat units of the phenolic compounds present in the composition.

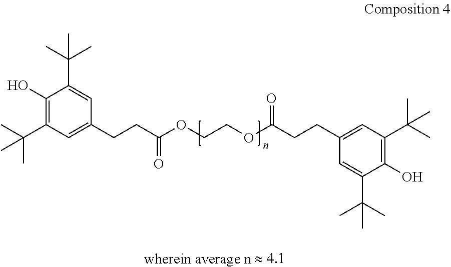

A 3-neck 100 mL round bottom flask was affixed with temperature probe, overhead stirring and a dean stark trap with a condenser under nitrogen atmosphere. To the reaction flask was charged, 23.4 g of methyl 3-[3,5-bis(tert-butyl)-4-hydroxyphenyl]propionate, 8 g of PEG 200 and 0.4 g dibutyltin oxide. The reaction was heated to 140° C. until complete as monitored by TLC. The product was a composition containing a distribution of phenolic compounds encompassed by the formula above, and the value of n is equal to the average number of ethylene oxide repeat units of the phenolic compounds present in the composition.

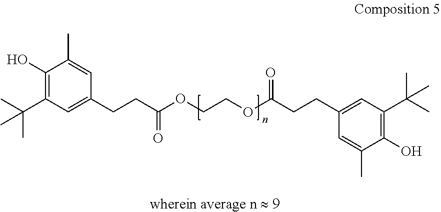

A 100 mL 3-neck round bottom flask was affixed with a temperature probe, overhead stirring and a dean-stark trap with a condenser under a nitrogen atmosphere. To the reaction flask was charged, 20 g of methyl 3-[3-(tert-butyl)-4-hydroxy-5-tolyl]propionate, 16 g of PEG 400 and 0.4 g sodium acetate. The reaction was heated to 100° C. and vacuum pulled for 1 hour to remove residual water. The reaction mixture was then heated to 160° C. and stirred until it was judged complete by TLC analysis. The product was cooled and transferred to a jar. The product was a composition containing a distribution of phenolic compounds encompassed by the formula above, and the value of n is equal to the average number of ethylene oxide repeat units of the phenolic compounds present in the composition.

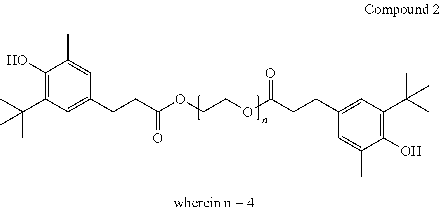

A 100 mL 3-neck round bottom flask was affixed with a temperature probe, overhead stirring and a dean-stark trap with a condenser under a nitrogen atmosphere. To the reaction flask was charged, 20 g of methyl 3-[3-(tert-butyl)-4-hydroxy-5-tolyl]propionate, 7.2 g of tetraethylene glycol (available from Sigma-Aldrich) and 0.4 g sodium acetate. The reaction was heated to 100° C. and vacuum pulled for 1 hour to remove residual water. The reaction mixture was then heated to 160° C. and stirred until it was judged complete by TLC analysis. The product was cooled and transferred to ajar.

### Example 2: Assessment of Malodor Reduction

In order to test the efficiency of the antioxidants for malodor reduction, an artificial body soil (ABS) was prepared. The artificial body soil is a modified version of the soil from ASTM D4265-21. To prepare the ABS, the components of Table 1 were combined in an 8 oz. jar and heated to 70° C. until fully solubilized.

TABLE 1
Artificial Body Soil Composition

| | Weight Percent |
|---|---|
| Material | Composition (%) |
| White coconut oil, refined (LouAna) | 7.5 |
| Stearic acid (TCI) | 2.5 |
| Squalene oil (Spectrum Chemical) | 2.5 |
| Cholesterol powder (TCI) | 2.5 |
| Olive oil (Bertolli) | 10 |
| Palmitic acid (TCI) | 5 |
| Paraffin oil (Spectrum Chemical) | 5 |
| Vegetable oil (GreatValue) | 10 |
| Oleic Acid (Emery Oleochemicals) | 5 |
| Dipropylene Glycol Monomethyl Ether (TCI) | 50 |

Fabric Preparation:

50/50 Cotton/poly interlock fabric (Style 7439 GB, Testfabrics, Inc.) was cut into 2 in×5 in swatches. Cut swatches were dosed with three equally spaced 0.36 mL portions of artificial body soil composition. Fabrics were then sealed together in a mylar bag and incubated at 37° C. for four to seven days prior to wash treatment.

Wash Water:

The wash water was used at 7 gpg hardness and 800 ng/mL of copper by dissolving the components listed in Table 2 in DI water.

TABLE 2
Wash Water Composition

| Component | Amount/L of Wash Water |
|---|---|
| Calcium Chloride | 0.0933 g |
| Magnesium Chloride | 0.0267 g |
| Copper (II) Chloride | 0.0017 g |

Antioxidant Solution Preparation:

Antioxidant solution was prepared by dissolving the antioxidant compound to be tested in methanol at 0.5% w/w. The antioxidant solution was dosed into the wash water (500 mL) using a micropipettor according to Table 3 to obtain the desired concentration of antioxidant in wash water required for the testing.

TABLE 3
Antioxidant Solution Addition to Wash Water and Corresponding
Concentration of Antioxidant in Wash Water

| Amount of antioxidant solution | Concentration of antioxidant |
|---|---|
| added to wash water | in wash water |
| 1.0 mL | 8 ppm |
| 0.5 mL | 4 ppm |
| 0.25 mL | 2 ppm |

Wash and Rinse Method:

To 1 L wash beaker was added 0.4 g Tide Free and Gentle detergent, 500 mL of wash water (as prepared in Table 2) and the antioxidant solution as described in Table 3. The wash beaker was placed in a tergetometer. To the wash beaker was added two incubated fabric swatches (4 g) followed by one 11 in×11 in 50/50 Cotton/Poly interlock knit clean fabric swatch (16 g, Style 74390B, Testfabrics, Inc.), for a total liquor to fabric ratio of 25:1. The fabrics were then washed in the tergotometer for 15 minutes and spun dried for one minute in a spin dryer (Mini Spin Dryer Model LASD-1 available from The Laundry Alternative, Inc.). Fabrics were then transferred from the spin dryer to 500 mL of fresh wash water (as prepared above) and rinsed for 15 minutes in the tergetometer. Fabrics were then spun dry for 1 minute in the spin dryer and transferred to a dryer to dry on medium heat for 1 hour. Once dried, the artificial body soil treated and washed fabrics were cut in half (1 in×5 in swatches), rolled, and put in gas chromatography vials for headspace analysis. The tests were run in triplicate and an average malodor reduction percent was calculated.

Malodor Analysis on Fabric Using GCMS Analysis

The GC data tracks the malodor marker concentration in the headspace that occurs from the oxidative breakdown of the ABS. The marker that is tracked for this breakdown of the ABS is 3-methyl-2-butenal.

The percent malodor reduction is determined by GCMS using an Agilent Intuvo 9000 GC equipped with an Agilent 7000D GC/TQ mass spectrometer and a Trajan PAL 3 RTC sampler equipped with a solid phase micro-extraction (SPME) probe. A calibration standard of the tracked malodor marker, 3-methyl-2-Butenal (107-86-8) is prepared by dissolving a known weight of mineral oil (CAS 8020-83-5). Fabrics are cut into uniform 1 inch by 5 inch pieces and placed in 10 mL headspace crimp vials. Vials are equilibrated greater than 12 hours before analysis. The following settings are used in the auto sampler: 90° C. incubation temperature, 90 min incubation time, VT25 sample tray type, 22 mm vial penetration, 10 min extraction time, 50 mm injection penetration and 3 min desorption time. The column is an Agilent DB-624 Ultra Inert, 60 m×250 μm×1.4 μm (Part number 122-1364UI-INT). The following settings are used for the Front Split/Splitless inlet helium: split mode, 250° C. temperature, 39.3 psi pressure, 30.5 mL/min total flow, 3 mL/min septum purge flow, 10:1 split ratio and 18.5 min GC run time. The follow settings are used in the oven: 35° C. initial temperature, hold 1 min at 35° C. then 30° C./min heating program, 260° C. final temperature and 10 min hold time at 260° C. The extracted ion chromatogram for 3-methyl-2-butenal includes Quantifier: 84, Qualifier: 55, Ratio: 35.7.

Results:

Following the test method as laid out above, the antioxidants were tested for the reduction of the malodor marker. All tests were run in triplicate and the average of the three swatches was taken. The difference in the counts between the control vs the antioxidant containing composition is used to calculate the percent malodor marker reduction. The percent malodor marker reduction was calculated by the following equation:

% malodor marker reduction=100−(Marker Amount_{Test}/Marker Amount_{Control}*100)

### Comparison of Substitution Pattern:

A comparison of the inventive compounds to antioxidants with di-tert-butyl substitutions on the phenolic rings of the antioxidants was made. Each antioxidant was run through the wash test at varying concentrations through the wash (2, 4, 8 ppm concentration of AO in wash water) and results are listed in Table 4. The preferred phenolic compositions of the invention (Compositions 1-3) are compared to the control (detergent nil antioxidant) and a comparative example (Composition 4) that does not contain the preferred substitution pattern on the hindered phenol moiety.

TABLE 4
Wash test results

| | | Molarity in | Average | |
|---|---|---|---|---|
| | Concentration AO | wash water | Malodor | % Malodor Marker |
| Sample | in wash water | (mols/L) | Marker Amount | Reduction |
| Control | 0 ppm | n/a | 13245581 | n/a |
| (detergent only | | | | |
| nil antioxidant) | | | | |
| Composition 1 | 8 ppm | 1.2 × 10 − 5 | 1228722 | 90.72 |
| Composition 1 | 4 ppm | 6.1 × 10 − 6 | 1663349 | 87.44 |
| Composition 2 | 2 ppm | 3.1 × 10 − 6 | 6714461 | 49.31 |
| Composition 2 | 8 ppm | 1.1 × 10 − 5 | 1621135 | 87.76 |
| Composition 2 | 4 ppm | 5.3 × 10 − 6 | 8066570 | 39.10 |
| Composition 2 | 2 ppm | 2.7 × 10 − 6 | 11581964 | 12.56 |
| Composition 3 | 8 ppm | 7.6 × 10 − 6 | 3760097 | 71.61 |
| Composition 3 | 4 ppm | 3.8 × 10 − 6 | 5551715 | 58.09 |
| Composition 3 | 2 ppm | 1.9 × 10 − 6 | 8818964 | 33.42 |
| Composition 4 | 8 ppm | 1.1 × 10 − 5 | 2465462 | 81.39 |
| Composition 4 | 4 ppm | 5.4 × 10 − 6 | 6405847 | 51.64 |
| Composition 4 | 2 ppm | 2.7 × 10 − 6 | 12259647 | 7.44 |

As one can see from the table above, all inventive phenolic compositions perform better than the control at reducing malodor and the preferred compositions, especially composition 1, which is the most preferred composition, perform as good or better than the comparative composition 4 at reducing the level of malodor marker.

### Comparison of Plurality of Molecules Vs Single Compound:

A comparison for malodor reduction of the inventive compositions to Irganox 245, which is a commercially available antioxidant from BASF that contains a 3EO chain linker as a single molecule, was made. Each antioxidant was run in a wash test at varying concentrations through the wash (2, 4, 8 ppm concentration of AO in wash water) and results are listed in Table 5. The preferred phenolic composition of the invention is compared to the control (detergent nil antioxidant) and a comparative example (Irganox 245) that does not contain a plurality of molecules was made.

TABLE 5
Wash test results

| | Concentration in | % Malodor |
|---|---|---|
| Sample | wash water | marker reduction |
| Control | n/a | n/a |
| (detergent only | | |
| nil antioxidant) | | |
| Composition 1 | 8 ppm | 89.4 |
| Composition 1 | 4 ppm | 80.7 |
| Composition 1 | 2 ppm | 51.0 |
| Irganox 245 | 8 ppm | 89.5 |
| Irganox 245 | 4 ppm | 74.4 |
| Irganox 245 | 2 ppm | 44.0 |

As one can see from the table above, the inventive phenolic composition 1 performs better than the control at reducing malodor and the preferred composition 1 performs better than Irganox 245, which is a single molecule, especially at lower concentrations in the wash water.

### Assessment of Stability

A comparison of the stability of the antioxidants in standard AATCC detergent was made to highlight the benefit of having a plurality of molecules. Due to the highly crystalline nature of many commercially available antioxidants, they tend to be incompatible in detergent and will crystallize out of the detergent over time. The inventive compounds of this invention show improvements in remaining stable in liquid detergent.

The antioxidants in table 6 were combined with AATCC HE liquid detergent without optical brighteners (available from Test Fabrics, Inc., Item #0501001) in a 20 mL glass scintillation vial. The vials were placed in a sonicator for 1 hour, then heated to 80° C. for 30 minutes, whereby all samples were completely dissolved. The samples were removed from the oven, swirled briefly and hand then allowed to cool to room temperature. When the vials were cooled down to room temperature, the bottom of the vials were scratched with a metal spatula a few times to aide in the crystallization process, which can take a long time if left undisturbed. After an additional 3 days, observations were recorded as crystallization present or not present in Table 6.

TABLE 6

| | Loading in AATCC | |
|---|---|---|
| Compound | detergent (ppm) | Precipitation Seen |
| Irganox 245 | 500 | No |
| | 1000 | No |
| | 5000 | Yes |
| | 10000 | Yes |
| Composition 1 | 500 | No |
| | 1000 | No |
| | 5000 | No |
| | 10000 | No |
| Composition 5 | 500 | No |
| | 1000 | No |
| | 5000 | No |
| | 10000 | No |
| Composition 3 | 500 | No |
| | 1000 | No |
| | 5000 | No |
| | 10000 | No |
| 50/50 blend of Irganox | 500 | No |
| 245 and Compound 6 | 1000 | No |
| | 5000 | No |
| | 10000 | No |

As one can see from the results in table 6, a plurality of molecules is beneficial to prevent precipitation of the antioxidant from the detergent formulation over time. This allows the formulator to incorporate higher levels of antioxidant into the detergent if desired and not be concerned about incompatibilities.

### Lower Concentration of Antioxidant in Detergent Test:

In order to show the malodor reduction efficiency of the inventive compositions at lower concentrations of phenolic antioxidant, a slight variation to the procedure was undertaken. The wash method was the same except adjustments to the copper loading in the wash water and the antioxidant use levels were made. For the experiment, the antioxidant level was reduced to 700 ppm in Tide Free and Gentle detergent (which equates to 0.56 ppm antioxidant concentration in wash water) and copper in wash water was used at a level of 100 ng Cu/g of wash water. Table 7 shows the results of the test.

TABLE 7
Wash test results with reduced copper and antioxidant levels

| | Concentration AO | % Malodor |
|---|---|---|
| Sample | in wash water | Marker Reduction |
| Control | 0 ppm | n/a |
| (detergent only | | |
| nil antioxidant) | | |
| Composition 1 | 0.56 ppm | 69.8 |
| Composition 2 | 0.56 ppm | 82.4 |
| Composition 3 | 0.56 ppm | 71.3 |

As one can see from Table 7, All inventive compositions perform well in reducing malodor markers on fabric with reduced loading levels through the wash.

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

What is claimed is:

1. A laundry care composition comprising:
a) a surfactant; and
b) a phenolic composition comprising:
iii) a first phenolic compound having the formula: 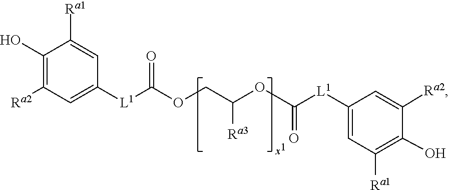
wherein L¹ is selected from null, —CH₂—, —CH₂CH₂—, and —CH₂CH₂CH₂—,
x¹ is an integer from 2-50;
R^{a1} is a C₁₋₈ alkyl group;
R^{a2} is a C₁₋₈ alkyl group;
R^{a3} is H, CH₃, or CH₂CH₃;
iv) a second phenolic compound having the formula: 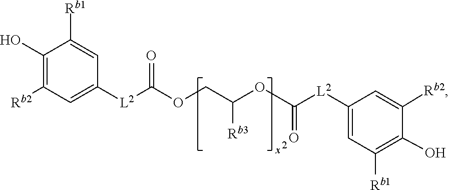
wherein L² is selected from null, —CH₂—, —CH₂CH₂—, and —CH₂CH₂CH₂—,
x² is an integer from 2-50; provided that x¹ and x² are not the same;
R^{b1} is a C₁₋₈ alkyl group;
R^{b2} is a C₁₋₈ alkyl group; and
R^{b3} is H, CH₃, or CH₂CH₃.

2. The laundry care composition according to claim 1, wherein the phenolic composition is present in an amount from 0.0001 wt. %-1.0 wt. % relative to the total weight of the laundry care composition.

3. The laundry care composition according to claim 1, wherein R^{a1} and R^{a2} are not the same.

4. The laundry care composition according to claim 1, wherein R^{b1} and R^{a2} are not the same.

5. The laundry care composition according to claim 1, wherein R^{a2} and R^{b2} are each C₃₋₅ alkyl.

6. The laundry care composition according to claim 5, wherein R^{a2} and R^{b2} are t-butyl.

7. The laundry care composition according to claim 1, wherein R^{a1} and R^{b1} are each a linear C₁₋₈ alkyl group.

8. The laundry care composition according to claim 7, wherein R^{a1} and R^{b1} are each a linear C₁₋₄ alkyl group.

9. The laundry care composition according to claim 8, wherein R^{a1} and R^{b1} are each methyl, or R^{a1} and R^{b1} are each ethyl.

10. The laundry care composition according to claim 1, wherein L¹ is —CH₂CH₂— or —CH₂CH₂CH₂—.

11. The laundry care composition according to claim 1, wherein L² is —CH₂CH₂— or —CH₂CH₂CH₂—.

12. The laundry care composition according to claim 1, wherein x¹ is an integer from 4-25.

13. The laundry care composition according to claim 1, wherein x² is an integer from 2-25.

14. The laundry care composition according to claim 1, wherein R^{a3} and R^{b3} are each H.

15. The laundry care composition according to claim 1, wherein the molar ratio of the first phenolic compound to the second phenolic compound is from 50:1 to 1:50.

16. The laundry care composition according to claim 1, further comprising a third phenolic compound having the formula: 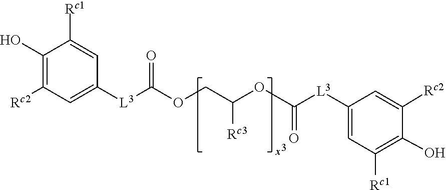
wherein L³ is selected from null, —CH₂—, —CH₂CH₂—, and —CH₂CH₂CH₂—,
x³ is an integer from 2-50; provided that none of x¹, x², and x³ are the same;
R^{c1} is a C₁₋₈ alkyl group;
R^{c2} is a C₁₋₈ alkyl group; and
R^{c3} is H, CH₃, or CH₂CH₃.

17. The laundry care composition according to claim 16, further comprising a fourth phenolic compound having the formula: 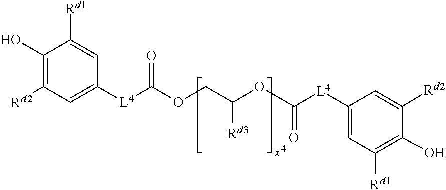
wherein L⁴ is selected from null, —CH₂—, —CH₂CH₂—, and —CH₂CH₂CH₂—,
x⁴ is an integer from 2-50; provided that none of x¹, x², x³, and x⁴ are the same;
R^{d1} is a C₁₋₈ alkyl group;
R^{d2} is a C₁₋₈ alkyl group; and
R^{d3} is H, CH₃, or CH₂CH₃.

18. A method of laundering an article comprising contacting the article with the composition according to claim 1.

19. The method according to claim 18, wherein the article is contacted with the composition in the presence of water.

20. A method of manufacturing a laundry care composition, comprising combining a water-soluble carrier, a surfactant and a phenolic composition, said phenolic composition comprising:
a first phenolic compound having the formula: 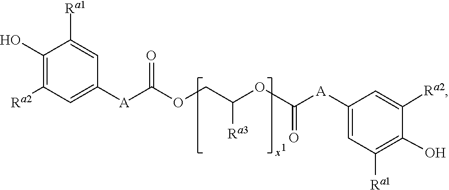
wherein A is selected from null, —CH₂—, —CH₂CH₂—, and —CH₂CH₂CH₂—,
x is an integer from 2-50;
R^{a1} is a C₁₋₈ alkyl group;
R^{a2} is a C₁₋₈ alkyl group;
R^{a3} is H, CH₃, or CH₂CH₃; and
a second phenolic compound having the formula: 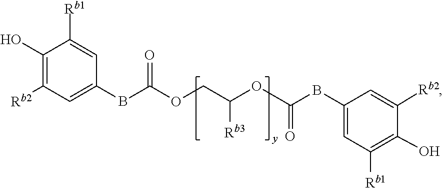
wherein B is selected from null, —CH₂—, —CH₂CH₂—, and —CH₂CH₂CH₂—,
y is an integer from 2-50; provided that x and y are not the same;
R^{b1} is C₁₋₈ alkyl group;
R^{b2} is a C₁₋₈ alkyl group; and
R^{b3} is H, CH₃, or CH₂CH₃;
